(19) United States
(12) Patent Application Publication
(10) Pub. No.: US 2010/0047820 A1
(43) Pub. Date: Feb. 25, 2010

(54) DETECTING METHOD, DETECTING APPARATUS, DETECTION SAMPLE CELL, AND DETECTING KIT

(75) Inventor: Hisashi Ohtsuka, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/546,147

(22) Filed: Aug. 24, 2009

(30) Foreign Application Priority Data
Aug. 25, 2008 (JP) 2008-215274

(51) Int. Cl. G01N 33/53; C12M 1/34

(52) U.S. Cl. 435/7.1; 435/288.7

(57) ABSTRACT

A sample is supplied onto the sensor portion of a sensor chip. An excitation light beam is irradiated to generate an enhanced optical field to be generated on the sensor portion. Fluorescent labels are excited, and the amount of a detection target substance is detected, based on the amount of light which is generated due to excitation of the fluorescent labels. A first electric charge is present on the surface of the sensor portion. A fluorescent substance having fluorescent pigment molecules which are enveloped in a light transmitting material that transmits fluorescence generated by the fluorescent pigment molecules, the surfaces of which are charged with second electric charges opposite the first electric charge on the surface are employed as the fluorescent labels. The fluorescent substance is attracted to the sensor portion by static electric interactions between the two charges.

### BACKGROUND OF THE INVENTION

1. Field of the Invention

The present invention is related to a detecting method, for detecting detection target substances within samples, a detection sample cell and a detecting kit.

2. Description of the Related Art

Fluorometry is conventionally used in biological measurements and the like, as an easy and highly sensitive measuring method. In fluorometry, a sample, which is considered to contain a detection target substance that emits fluorescence when excited by light having a specific wavelength, is irradiated with an excitation light beam of the aforementioned specific wavelength. The presence of the detection target substance can be confirmed quantitatively by detecting the fluorescence due to the excitation. In the case that the detection target substance is not a fluorescent substance, the detection target sample is labeled with fluorescent labels, such as organic fluorescent pigment. Thereafter, fluorescence is detected in the same manner as described above, thereby confirming the presence of the detection target substance, by the presence of the fluorescent labels.

In fluorometry, it is common to employ one of the two methods described below to immobilize detection target substances onto the surfaces of sensor portions and then to detect fluorescence, for the reasons that they enable efficient detection of only the detection target substances while causing a sample to flow. The first of the two methods, the so called “sandwich method” will be described for a case that the detection target substance is an antigen, as an example. In the sandwich method, the antigens are caused to specifically bind to primary antibodies which are immobilized onto the surface of a sensor portion. Next, secondary antibodies that specifically bind to the antigens and which have fluorescent labels attached thereto are caused to bind with the antigens, thereby forming a binding state among the primary antibodies, the antigens, and the secondary antibodies. Thereafter, fluorescence emitted by the fluorescent labels which are attached to the secondary antibodies is detected. The second of the two methods, the so called “competition method”, will also be described for a case that the detection target substance is an antigen, as an example. In the competition method, the antigens and secondary antibodies, to which fluorescent labels are attached (these secondary antibodies are different from the aforementioned secondary antibodies, and specifically bind to primary antibodies) are caused to bind with primary antibodies which are immobilized on to the surface of a sensor portion in a competitive manner. Thereafter, fluorescence emitted by the fluorescent labels which are attached to the secondary antibodies is detected.

Evanescent fluorometry, in which fluorescent labels, which are indirectly immobilized onto sensor portions by the methods described above, are excited by evanescent light, has been proposed for the reason that S/N ratios can be improved. In evanescent fluorometry, an excitation light beam is caused to enter a sensor portion from the rear surface thereof. Fluorescent labels are excited by evanescent light that leaks onto the front surface of the sensor portion. Thereafter, fluorescence emitted by the fluorescent labels is detected.

Meanwhile, a method that utilizes the electric field enhancing effect of plasmon resonance in order to improve the sensitivity of evanescent fluorometry has been proposed in U.S. Pat. No. 6,194,223 and M. M. L. M. Vareiro et al., “Surface Plasmon Fluorescence Measurements of Human Chorionic Gonadotrophin: Role of Antibody Orientation in Obtaining Enhanced Sensitivity and Limit of Detection”, Analytical Chemistry, Vol. 77, No. 8, pp. 2426-2431, 2005. In this surface plasmon enhanced fluorometry method, a metal layer is provided at a sensor portion, surface plasmon is generated at the metal layer, and fluorescence signals are amplified by the electric field enhancing effects of the surface plasmon, to improve the S/N ratio.

In addition, a method in which the electric field enhancing effects of an optical waveguide mode is utilized in order to enhance the electric field at the sensor portion, similarly to the surface plasmon enhanced fluorometry method, has been proposed in K. Tsuboi et al., “High-sensitive sensing of catechol amines using by optical waveguide mode enhanced fluorescence spectroscopy”, 54^{th} Meeting of the Japan Society of Applied Physics, Collection of Presentation Abstracts, No. 3, p. 1378, 28p-SA-4, Spring 2007. In optical waveguide mode enhanced fluorescence spectroscopy, a metal layer and an optical waveguide layer constituted by a dielectric or the like are sequentially provided on a sensor portion. An optical waveguide mode is generated in the optical waveguide layer, and fluorescence signals are amplified by the electric field enhancing effects thereof.

In addition, U.S. Patent Application Publication No. 20050053974 and T. Liebermann and W. Knoll, “Surface-plasmon field-enhanced fluorescence spectroscopy”, Colloids and Surfaces A, Vol. 171, pp. 115-130, 2000, propose methods for detecting radiant light (SPCE: Surface Plasmon Coupled Emission), which is generated by surface plasmon induced at metal layers by fluorescence generated by fluorescent labels, instead of detecting fluorescence emitted by fluorescent labels as in the aforementioned fluorometry methods.

As described above, various methods for generating plasmon resonance or optical waveguide modes by the irradiation of excitation light beams, exciting fluorescent labels by the optical fields enhanced by the plasmon resonance or optical waveguide modes, and detecting light generated due to the excitation of the fluorescent labels have been proposed in order to detect detection target substances in biological measurement.

However, the electric field enhancing effects of surface plasmon resonance and optical waveguide modes attenuate drastically as distances from the surfaces of metal layers and optical waveguide layers increase. Therefore, differences occur in signals by slight changes in distance between the surfaces and fluorescent labels, resulting in a problem that signal fluctuations occur.

FIG. 18 is a schematic diagram that illustrates the vicinity of a sensor portion of an apparatus that detects fluorescence which is amplified by the electric field enhancing effect of surface plasmon resonance. A metal film 102 is provided on a prism 101 (substrate), and primary antibodies B₁ are immobilized onto the metal film 102. In the case that a sandwich method assay is performed, binding states are formed among the primary antibodies B₁, antigens A, and labeling secondary antibodies B₂. Here, the labeling secondary antibodies B₂ have fluorescent labels (here, fluorescent pigment molecules f) attached thereto. Surface plasmon is excited at the surface of the metal film 102, by causing an excitation light beam to enter the interface between the prism 101 and the metal film 102 at an angle greater than or equal to a total reflection angle. Thereby, surface plasmon is excited at the surface of the metal film 102, and the optical field at the surface of the metal film is enhanced. As a result, the fluorescent labels f are excited by the enhanced optical field, and emit fluorescence.

The graph of FIG. 18 illustrates the dependence of the intensity of the electric field on the distance from the surface of a sensor portion (the surface of the metal film 102). As illustrated in the graph, the electric field intensity drastically attenuates as the distance from the surface of the metal film 102 increases. There are cases in which the distance from the surface of the sensor portion to the fluorescent labels f of the labeling secondary antibodies B₂ become as great as 50 nm. In these cases, the fluorescent intensity attenuates by 30% or greater. In addition, the primary antibodies B₁ are not always immobilized onto the surface of the sensor portion in an upright state, and there are cases in which the flow of liquids or three dimensional obstacles cause the primary antibodies B₁ to collapse along the surface. Accordingly, this causes fluctuations in the distances between the surface of the sensor portion and the fluorescent labels f, which leads to fluctuations in signal intensities.

### SUMMARY OF THE INVENTION

The present invention has been developed in view of the foregoing circumstances. It is an object of the present invention to provide a detecting method, and a detecting apparatus that enable detection of light generated due to excitation of fluorescent labels that suppresses fluctuations in signal intensities, while efficiently utilizing enhanced electric fields.

It is another object of the present invention to provide a detection sample cell and a detecting kit to be employed in the above detecting method.

A detecting method of the present invention comprises the steps of:

employing a sensor chip having a sensor portion provided on a surface of a dielectric plate;

causing a sample to contact the sensor portion, to cause an amount of a fluorescent label binding substance corresponding to the amount of a detection target substance included in the sample to bind onto the sensor portion;

irradiating an excitation light beam onto the sensor portion, to cause an enhanced optical field to be generated thereon;

exciting fluorescent labels of the fluorescent label binding substance with the enhanced optical field; and

detecting the amount of the detection target substance, based on the amount of light which is generated due to excitation of the fluorescent labels;

the sensor chip being that in which the sensor portion is of a laminated structure including a metal layer adjacent to the dielectric plate, and a first electric charge being present on the surface of the sensor portion; and

a fluorescent substance having fluorescent pigment molecules which are enveloped in a light transmitting material that transmits fluorescence generated by the fluorescent pigment molecules, the surfaces of which are charged with second electric charges opposite the first electric charge being employed as the fluorescent labels.

Here, the term “laminated structure including a metal layer” refers to a structure that includes a metal layer and is constituted by at least one layer. That is, the laminated structure may consist of only the metal layer. Note that the “layer” refers not only to structures that cover the entirety of a surface, but also those that have fine apertures or surface roughness.

The term “fluorescent label binding substance” refers to a binding substance which are labeled with fluorescent labels and bind to the sensor portion in an amount corresponding to the amount of the detection target substance. In the case that assays are to be performed by the sandwich method, the term “fluorescent label binding substance” refers to a binding substance that specifically binds with the detection target substance and fluorescent labels. On the other hand, in the case that assays are to be performed by the competition method, the term “fluorescent label binding substance” refers to a binding substance that competes with the detection target substance and fluorescent labels.

The term “light which is generated due to excitation of the fluorescent labels” is light which is generated either directly or indirectly by the excitation of the fluorescent labels. The term “light which is generated due to excitation of the fluorescent labels” refers to light of which the generated amount has a correlation with the number of excited fluorescent labels.

The phrase “detecting the amount of the detection target substance” refers also to detecting whether the detection target substance is present in the sample. That is, the phrase refers both to quantitative detection and qualitative detection.

The term “optical field” refers to an electric field generated by evanescent light or near field light, which is generated due to irradiation of the excitation light beam.

The phrase “to cause an enhanced optical field to be generated” refers to forming an enhanced optical field, by enhancing a optical field. Note that the method by which the optical field is enhanced may be enhancement due to excitement of plasmon resonance, or enhancement due to excitement of an optical waveguide mode.

A single fluorescent pigment molecule may be enveloped in the light transmitting material, but it is preferable for a plurality of fluorescent pigment molecules to be enveloped in the light transmitting material. Note that in the case that the fluorescent substance includes a plurality of fluorescent pigment molecules, a portion of the fluorescent pigment molecules may be exposed to the exterior of the light transmitting, as long as at least one fluorescent pigment molecule is enveloped therein.

In the detecting method of the present invention, it is preferable for the first electric charge to be imparted by a functional group, which is one of an acidic functional group and a basic functional group, corresponding to the charge; and for the second electric charges to be imparted by the other of the acidic functional group and the basic functional group.

It is also preferable for the intensities of the first and second electric charges to be controlled by controlling the pH of a solution on the sensor portion.

It is preferable for the outermost surface layer of the laminated structure of the sensor chip to be constituted by a self assembling film; and for functional groups which are included in the self assembling film are employed as the functional group that imparts the first electric charge onto the surface of the sensor portion. Alternatively, the outermost surface layer of the laminated structure of the sensor chip may be constituted by a polymer compound film; and functional groups which are included in the polymer compound film may be employed as the functional group that imparts the first electric charge onto the surface of the sensor portion. In this case, it is preferable for a blocking agent to be employed as the polymer compound film.

Further, “exciting fluorescent labels of the fluorescent label binding substance with the enhanced optical field; and detecting the amount of the detection target substance, based on the amount of light which is generated due to excitation of the fluorescent labels” may be performed by detecting the fluorescence emitted by the fluorescent labels, or by detecting radiant light, which is generated by plasmon induced at the metal layer by the fluorescence. Specifically, any one of the following methods (1) through (4) may be employed.

(1) Exciting plasmon at the metal layer by irradiating the excitation light beam, and the enhanced optical field is generated by the plasmon; and detecting fluorescence generated by the fluorescent labels due to excitation as the light which is generated due to the excitation of the fluorescent labels.

(2) Exciting plasmon at the metal layer by irradiating the excitation light beam, and the enhanced optical field is generated by the plasmon; and detecting radiant light that radiates toward the surface of the dielectric plate opposite the surface on which the sensor portion is provided from plasmon newly induced in the metal layer by fluorescence generated by the fluorescent labels due to excitation, as the light which is generated due to the excitation of the fluorescent labels.

(3) Employing a sensor chip having a laminated structure equipped with an optical waveguide layer; inducing an optical waveguide mode within the optical waveguide layer by irradiating the excitation light beam; generating the enhanced optical field by the optical waveguide mode; and detecting fluorescence generated by the fluorescent labels due to excitation as the light which is generated due to the excitation of the fluorescent labels.

(4) Employing a sensor chip having a laminated structure equipped with an optical waveguide layer; inducing an optical waveguide mode within the optical waveguide layer by irradiating the excitation light beam; generating the enhanced optical field by the optical waveguide mode; and detecting radiant light that radiates toward the surface of the dielectric plate opposite the surface on which the sensor portion is provided from plasmon induced at the metal layer by fluorescence generated by the fluorescent labels due to excitation, as the light which is generated due to the excitation of the fluorescent labels.

Note that in the aforementioned methods (1) and (2), the excitation of plasmon may be realized by employing a metal film as the metal layer, causing the excitation light beam to enter the interface between the metal film and the substrate at an angle greater than or equal to a total reflection angle from the rear surface of the substrate, to excite surface plasmon on the surface of the metal film. Alternatively, the plasmon may be excited by employing a finely structured metal piece having recesses and protrusions at a frequency smaller than the wavelength of the excitation light beam, or metal nano rods of sizes smaller than the wavelength of the excitation light beam as the metal layer, and causing localized plasmon to be excited by the irradiation of the excitation light beam.

A detecting apparatus according to the present invention is an apparatus to be employed to execute the detecting method of the present invention and comprises:

a housing portion for housing the sensor chip;

an excitation light beam irradiating optical system, for irradiating the excitation light beam onto the sensor portion;

light detecting means, for detecting the light which is generated due to excitation of the fluorescent labels by the enhanced optical field; and

pH control means, for controlling the pH of solutions on the sensor portion of the sensor chip which is housed in the housing portion.

A detection sample cell according to the present invention is a detection sample cell to be utilized in the detecting method of the present invention, and comprises:

a base having a channel through which liquid samples are caused to flow;

an injection opening provided at an upstream side of the channel for injecting the liquid samples into the channel;

an air aperture provided at a downstream side of the channel for causing the liquid samples which have been injected from the injection opening to flow downstream; and

a sensor chip portion provided within the channel between the injection opening and the air aperture, comprising a dielectric plate which is provided as a portion of an inner wall of the channel, and a sensor portion provided on a predetermined region of the dielectric plate on the sample contacting surface thereof;

the sensor portion being of a laminated structure that includes a metal layer adjacent to the dielectric plate; and

one of an acidic functional group and a basic functional group being present on the surface of the sensor portion.

It is preferable for the detection sample cell of the present invention to further comprise a first binding substance, for immobilizing the fluorescent label binding substance onto the sensor portion, immobilized onto the sensor portion.

Alternatively, it is preferable for the detection sample cell of the present invention to further comprise: one of a second binding substance that specifically binds with the detection target substance and a third binding substance that specifically binds with the first binding substance and competes with the detection target substance; and a fluorescent label binding substance modified with one of the second binding substance and third binding substance, having the one of an acidic functional group and a basic functional group, which is different from the functional group provided on the sensor portion, provided in the channel upstream from the sensor portion.

Note that in the case that the fluorescent label binding substance is constituted by the second binding substance and the fluorescent labels, the detection sample cell of the present invention is favorable for use in performing assays by the sandwich method. Alternatively, in the case that the fluorescent label binding substance is constituted by the third binding substance and the fluorescent labels, the detection sample cell of the present invention is favorable for use in performing assays by the competition method.

In the detection sample cell of the present invention, it is preferable for the laminated structure to be equipped with an optical waveguide layer.

A detecting kit according to the present invention is a detecting kit to be utilized to execute the detecting method of the present invention, and comprises:

a detection sample cell equipped with: a base having a channel through which liquid samples are caused to flow; an injection opening provided at an upstream side of the channel for injecting the liquid samples into the channel; an air aperture provided at a downstream side of the channel for causing the liquid samples which have been injected from the injection opening to flow downstream; a sensor chip portion provided within the channel between the injection opening and the air aperture, comprising a dielectric plate which is provided as a portion of an inner wall of the channel, and a sensor portion provided on a predetermined region of the dielectric plate on the sample contacting surface thereof; a first binding substance for immobilizing the fluorescent label binding substance onto the sensor portion, immobilized onto the sensor portion; the sensor portion being of a laminated structure that includes a metal layer adjacent to the dielectric plate; and one of an acidic functional group and a basic functional group being present on the surface of the sensor portion; and

a labeling solution which is caused to flow into the channel at one of a timing simultaneously with the liquid sample and a timing after the liquid sample is caused to flow into the channel, including a fluorescent substance modified with one of: a second binding substance that specifically binds with the detection target substance and a third binding substance that specifically binds with the first binding substance and competes with the detection target substance.

Note that in the case that the fluorescent label binding substance is constituted by the second binding substance and the fluorescent labels, the detecting kit of the present invention is favorable for use in performing assays by the sandwich method. Alternatively, in the case that the fluorescent label binding substance is constituted by the third binding substance and the fluorescent labels, the detecting kit of the present invention is favorable for use in performing assays by the competition method.

In the detecting kit of the present invention, it is preferable for the laminated structure to be equipped with an optical waveguide layer.

In the detection sample cell and the detecting kit of the present invention, the “fluorescent substance” is that in which fluorescent pigment molecules are enveloped in a light transmitting material that transmits fluorescence generated by the fluorescent pigment molecules.

In the detecting method and the detecting apparatus of the present invention, detection is performed in a state in which a first electric charge is imparted to the sensor portion, and a second electric charge opposite the first electric charge is imparted onto the fluorescent substance that functions as fluorescent labels. Accordingly, the fluorescent labels are drawn to the vicinity of the surface of the sensor portion, at which the electric field enhancing effect is great, due to static electric interactions. Therefore, amounts of light which are generated due to excitation of the fluorescent labels in a state that the fluorescent labels are in the vicinity of the surface of the sensor portion can be detected. As a result, the electric field at the surface of the sensor portion, at which the degree of enhancement is great, can be efficiently utilized, and the distances from the surface of the sensor portion to the fluorescent labels can be uniformized. Accordingly, fluctuations in signal intensities can be suppressed. That is, stable signals having favorable S/N ratios can be detected, and the presence and/or the amounts of detection target substances can be accurately detected.

If the detection sample cell or the detecting kit of the present invention is employed, the detecting method of the present invention can be easily executed. Therefore, the enhanced electric field can be effectively utilized while fluctuations in signal intensities can be suppressed, and the presence and/or the amounts of detection target substances can be accurately detected.

### BRIEF DESCRIPTION OF THE DRAWINGS

FIG. 1 is a schematic diagram that illustrates a detecting apparatus (SPF) according to a first embodiment of the present invention.

FIG. 2A is a first diagram that schematically illustrates a charged state in the vicinity of a sensor portion.

FIG. 2B is a second diagram that schematically illustrates a charged state in the vicinity of a sensor portion.

FIG. 3 is a diagram that illustrates a design modification to an excitation light irradiating optical system.

FIG. 4 is a schematic diagram that illustrates the construction of a fluorescence detecting apparatus (LPF) according to a second embodiment of the present invention.

FIG. 5A, FIG. 5B, and FIG. 5C are schematic diagrams that illustrate examples of sensor chips which are employed by the fluorescence detecting apparatus according tot eh second embodiment of the present invention.

FIG. 6 is a schematic diagram that illustrates the construction of a detecting apparatus (SPCE) according to a third embodiment of the present invention.

FIG. 7 is a schematic diagram that illustrates the construction of a detecting apparatus (waveguide-fluorescence) according to a fourth embodiment of the present invention.

FIG. 8 is a schematic diagram that illustrates the construction of a detecting apparatus (waveguide-radiant light) according to a fifth embodiment of the present invention.

FIG. 9 is a schematic diagram that illustrates a fluorescent substance having a metal coating film.

FIG. 10A is a plan view that illustrates the construction of a detection sample cell according to a first embodiment of the present invention.

FIG. 10B is a cross sectional side view of the detection sample cell according to the first embodiment of the present invention.

FIG. 11 is a side view of a detection sample cell according to a second embodiment of the present invention.

FIG. 12 is a diagram that illustrates the steps of an assay which is performed according to the sandwich method, employing the detection sample cell of the second embodiment.

FIG. 13 is a side view of a detection sample cell according to a third embodiment of the present invention.

FIG. 14 is a diagram that illustrates the steps of an assay which is performed according to the competition method, employing the detection sample cell of the third embodiment.

FIG. 15 is a sectional side view that illustrates a design modification to the detection sample cell of the present invention.

FIG. 16 is a schematic diagram that illustrates a detecting kit according to the present invention.

FIG. 17 is a diagram that illustrates the steps of an assay which is performed according to the sandwich method, employing the detecting kit of the present invention.

FIG. 18 is a conceptual diagram that illustrates a conventional detecting method.

### BEST MODE FOR CARRYING OUT THE INVENTION

Hereinafter, embodiments of the present invention will be described with reference to the attached drawings. However, the present invention is not limited to the embodiments to be described below.

### [Detecting Method and Detecting Apparatus]

### First Embodiment

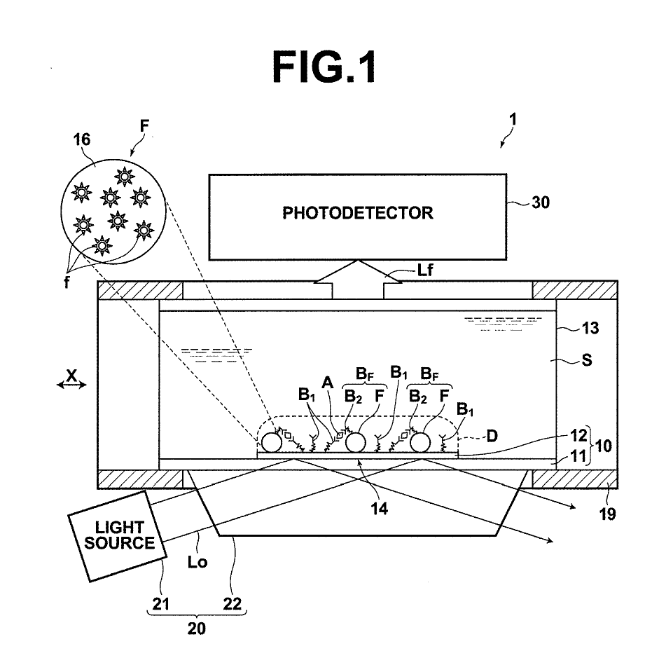

A detecting method of the present invention employs a sensor chip 10 having a sensor portion 14 that includes a metal layer 12 provided on a surface of a dielectric plate 11 as illustrated in FIG. 1. A sample is caused to contact the sensor portion 14, to cause an amount of a fluorescent label binding substance B_{F} corresponding to the amount of a detection target substance A included in the sample to bind onto the sensor portion 14. Then, an excitation light beam Lo is irradiated onto the sensor portion 14, to enhance an optical field on the sensor portion 14. A fluorescent substance F of the fluorescent label binding substance B_{F} is excited with the enhanced optical field D, and the amount of the detection target substance A is detected, based on the amount of light which is generated due to excitation of the fluorescent substance F. Positive electric charges are imparted on the surface of the sensor portion 14, and negative electric charges are imparted onto the surfaces of the fluorescent substance F. Thereby, detection of the detection target substance A is performed in a state in which the fluorescent substance F is drawn to the vicinity of the sensor portion 14.

A detecting apparatus of the present invention is employed to execute the detecting method above, and comprises: a housing portion 19 for housing the sensor chip 10; an excitation light beam irradiating optical system 20 for irradiating the excitation light beam Lo onto the sensor portion 14; a photodetector 30 for detecting amounts of light that correspond to amounts of the detection target substance A; and a pH control means (not shown) for controlling the pH of solutions on the sensor portion 14 of the sensor chip 10 which is housed in the housing portion 19.

In the present invention, the enhanced optical field D is generated on the sensor portion 14 by the irradiation of the excitation light beam Lo, and light which is generated due to excitation of fluorescent labels by the enhanced optical field D is detected. The method by which the optical field D is enhanced may be by surface plasmon resonance, localized plasmon resonance, or by excitation of an optical waveguide mode (fourth embodiment). In addition, fluorescence Lf emitted by the fluorescent labels may be detected, or radiant light Lp, which is generated due to surface plasmon excited on the metal layer 12 by the fluorescence Lf, may be detected (third embodiment). The details will be described in each of the following embodiments. Note that the detecting method and the detecting apparatus of the first embodiment enhances the optical field D by surface plasmon resonance, and detects fluorescence Lf, which is excited by the enhanced optical field D.

In the detecting method of the first embodiment, the sensor portion of the sensor chip 10 to be employed is equipped with a dielectric plate 11 and the metal layer 12, which is provided at a predetermined region on a surface of the dielectric plate 11.

The sensor chip 10 is constituted by the metal layer 12, which is formed as a metal film on a predetermined region on the surface of the dielectric plate 11, which is a glass plate or the like. The dielectric plate 11 may be formed by transparent materials such as transparent resins and glass. It is desirable for the dielectric plate 11 to be formed by resin. In the case that the dielectric plate 11 is formed by resin, polymethyl methacrylate (PMMA), polycarbonate (PC), and non crystalline polyolefin (APO) that includes cycloolefin may be favorably employed. The metal layer 12 is formed on the surface of the dielectric plate 11 using a mask having an opening at the predetermined region. The metal layer 12 may be formed by known film forming methods such as the vapor deposition method and the sputtering method. It is preferable for the thickness of the metal layer 12 to be determined such that surface plasmon is strongly excited, taking the material of the metal layer 12a and the wavelength of the excitation light beam Lo into consideration. For example, in the case that a laser beam having a central wavelength of 780 nm is employed as the excitation light beam Lo, and an Au film is employed as the metal layer 12, a favorable thickness of the metal layer 12a is 50 nm±20 nm. In this case, it is more preferable for the thickness of the metal layer 12 to be 47 nm±10 nm. Note that it is preferable for the metal layer 12 to be a metal having at least one of Au, Ag, Cu, Al, Pt, Ni, Ti, and alloys thereof as a main component. Here, the term “main component” is defined as a component which is included in the metal at 90 weight % or greater.

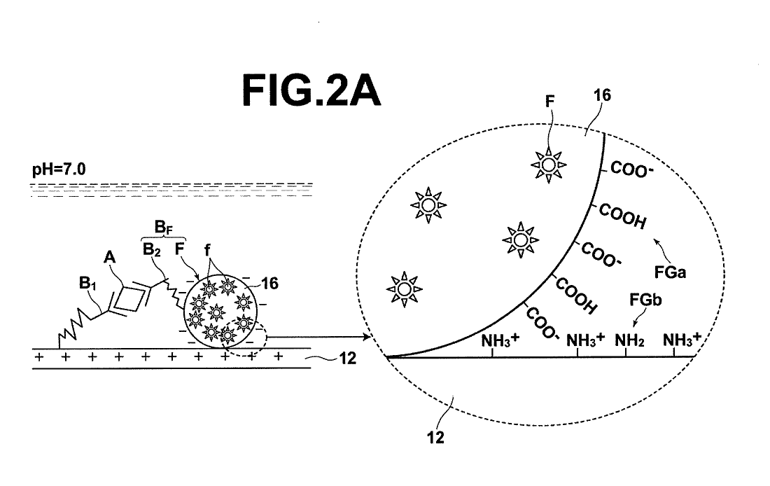

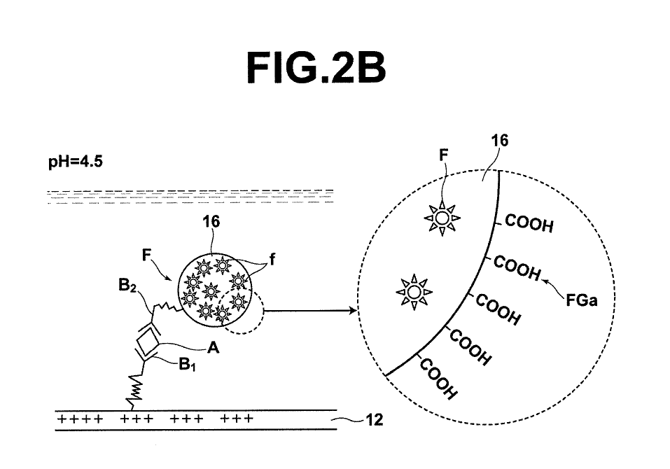

Further, in the first embodiment, basic functional groups are immobilized on the surface of the metal film 12, in order to charge the surface with positive electric charges. In FIG. 2A, amino groups are illustrated as examples of a basic functional group. However, the basic functional group is not particularly limited, and quaternary ammonium groups and the like may be employed. Note that the sign of the electric charges is also not particularly limited, and acidic functional groups may be immobilized on the surface of the metal film 12 in order to charge the surface with negative electric charges. Examples of acidic functional groups to be employed in this case include: carboxyl groups; sulfonic acid groups; and phosphoric acid groups. For example, carboxyl groups (—COOH) may charge the fluorescent substance F with negative charges, by becoming ionized into COO⁻ within liquid samples such as blood serum and plasma, or PBS (Phosphate Buffered Saline). As another example, amino groups (—NH₂) may charge the fluorescent substance F with positive charges, by becoming ionized into NH₃⁺ within liquid samples such as blood serum and plasma, or PBS. The method by which the functional groups are immobilized onto the surface of the metal film 12 is not particularly limited. However, it is preferable for the functional groups to be immobilized by employing self assembling films or polymer compound films that have the functional groups.

A method by which the functional groups are immobilized by employing a self assembling film having the functional groups will be described. Sulfur compounds such as thiols and disulfides actively adsorb onto precious metal substrates such as gold, and impart super thin films of single molecule sizes. Agglomerations of the sulfur compounds exhibit arrangements dependent on the crystal lattices of the substrate and the molecular structures of the adsorbing molecules, and therefore are referred to as self assembled films. Examples of self assembling films include alkanethiols on gold surfaces, alkyl silanes on glass surfaces, and alcohols on silicon surfaces. Specific examples of alkanethiols include: 7-carboxy-1-heptanethiol; 1,0-carboxy-1-decanethiol; 4, 4′-dithio dibutylic acid; 1,1-hydroxy-1- undecanethiol; and 1,1-amino-1-undecanethiol. Note that the material to be employed may be appropriately selected, taking the electric charges to be imparted into consideration.

A method by which the functional groups are immobilized by employing a polymer compound film having the functional groups will be described. Hydrophobic polymer compounds or water soluble polymer compounds are preferable as the polymer compound to be utilized in the present invention. Specific examples of hydrophobic polymer compounds include: polyacrylic acid derivatives; polymethacrylic acid derivatives; polyethylene (PE); polypropylene (PP); polybutadiene; polymethyl pentene; cycloolefin polymer; polystyrene (PS); acrylonitryl/butadiene/styrene copolymer (ABS); styrene/maleic acid anhydride copolymer; polyvinyl chloride (PVC); polyethylene terephthalate (PET); polyethylene naphthalate (PEN); nylon 6; nylon 6·6; cellulose acetate (TAC); polycarbonate (PC); modified phlyphenylene ether (m-PPE); polyphenylene sulfide (PPS); polyether ketone (PEK); polyether ether ketone (PEEK); polysulfone (PS); polyether sulfone (PES); polyphenylene sulfide (PPS); and liquid crystal polymer (LCP). Specific examples of water soluble polymer compounds include: natural polymers such as dextran derivatives, starch derivatives, cellulose derivatives, and gelatin; and synthetic polymers such as polyvinyl alcohol, polyethylene glycol, polyvinyl pyrolidone, polyacryl amide derivatives, and polymethyl vinyl ether. The method by which acidic functional groups are introduced into the polymer compound is not particularly limited. Copolymers may be produced by copolymerization reactions among monomeric molecules and monomeric molecules having the acidic functional groups. Alternatively, copolymers may be produced in advanced, then the acidic functional groups may be introduced by polymerization reactions. The polymer compounds to be employed in the present invention may be copolymers with monomeric molecules other than those that have the acidic functional groups. Note that the material to be employed may be appropriately selected, taking the electric charges to be imparted into consideration.

Further, a blocking agent which is employed in biosensing may be employed as the polymer compound. Examples of blocking agents include blood serum albumin (BSA) and casein, which exhibit negative electric charging properties under neutral conditions.

The fluorescent label binding substance B_{F} is a binding substance which are labeled with fluorescent labels and bind to the sensor portion 14 in an amount corresponding to the amount of the detection target substance A. In the case that assays are to be performed by the sandwich method as illustrated in FIG. 1, the fluorescent label binding substance B_{F} is constituted by a second binding substance B₂ that specifically binds with the detection target substance A and fluorescent labels. On the other hand, in the case that assays are to be performed by the competition method to be described later, the fluorescent label binding substance B_{F} is constituted by a third binding substance that competes with the detection target substance A and fluorescent labels. More specifically, in the case that a sensor chip 10 having a first binding substance B₁ that specifically binds with the detection target substance A immobilized on the sensor portion 14 is employed and assays are to be performed by the sandwich method, the fluorescent binding substance B_{F} is constituted by the second binding substance B₂, and fluorescent labels which are modified with the second binding substance B₂. On the other hand, in the case that the same sensor chip 10 is employed to perform assays by the competition method, the fluorescent binding substance B_{F} is constituted by a third binding substance that compete with the detection target substance to specifically bind with the first binding substance B₁, and fluorescent labels which are modified with the third binding substance.

As illustrated in the magnified portion of FIG. 1, the fluorescent substance F is that in which a plurality of fluorescent pigment molecules f are enveloped in a light transmitting material 16 that transmits fluorescence generated by the fluorescent pigment molecules f. The fluorescent substance F may be produced in the following manner.

First, a 0.1% solid in phosphate polystyrene solution having a pH of 7.0 and that contains polystyrene particles (product number K1-050 by Estapor, φ500 nm, 10% solid, carboxyl base) is produced.

Next, 1 ml of a phosphate ethyl solution containing 0.3 mg of fluorescent pigment molecules (NK-2014 by Hayashibara Biochemical, excitation wavelength: 780 nm) is produced.

The polystyrene solution and the fluorescent pigment solution are mixed, and impregnation is performed while evaporating the mixture. Then, the mixture is placed in a centrifuge (15000 rpm, 4° C., 20 minutes, two times), and supernatant liquid is removed.

The foregoing steps result in obtainment of the fluorescent substance F, in which polystyrene that functions to transmit fluorescence emitted from the fluorescent pigment molecules f has the fluorescent pigment molecules f enveloped therein. The particle size of each particle of the fluorescent substance F which is produced by impregnating the polystyrene particles with the fluorescent pigment molecules f is the same as the particle size of the polystyrene particles (φ500 nm in the example above).

Each particle of the fluorescent substance F has a plurality of fluorescent pigment molecules f enveloped in a light transmitting material 16. Therefore, the amount of emitted fluorescence can be greatly increased compared to conventional cases in which single fluorescent pigment molecules f are employed as fluorescent labels. Note that it is preferable for the particle size of each particle of the fluorescent substance F to be less than or equal to 5300 nm, and more preferable for the particle size of the fluorescent substance to be within a range from 100 nm to 700 nm. The most preferred range of article sizes for each particle of the fluorescent substance F is within a range from 130 nm to 500 nm. Specific examples of the light transmitting material 16 include polystyrene and SiO₂. However, the light transmitting material 16 is not particularly limited, as long as it is capable of enveloping the fluorescent pigment molecules f and transmitting the fluorescence Lf emitted by the fluorescent pigment molecules f toward the exterior.

Further, in the first embodiment, acidic functional groups are immobilized on the surfaces of the fluorescent substance F in order to charge the surfaces with negative electric charges, as illustrated in FIG. 2A. In FIG. 2A, carboxyl groups are illustrated as examples of the acidic functional group. However, the acidic functional group is not particularly limited, and other acidic functional groups, such as sulfonic acid groups and phosphoric acid groups may be employed. Note that the sign of the electric charges is also not particularly limited, and basic functional groups may be immobilized on the surfaces of the fluorescent substance F in order to charge the surfaces with positive electric charges. Examples of basic functional groups to be employed in this case include: amino groups and quaternary ammonium groups. The method by which the functional groups are immobilized is also not particularly limited, and known techniques may be employed. For example, carboxyl groups may be introduced to the surfaces of the fluorescent substance F by copolymerization using carbonic acid monomers.

Note that in the first embodiment, the sensor chip 10 is equipped with a sample holding portion 13 for holding liquid samples S. The sensor chip 10 and the sample holding portion 13 constitute a box shaped cell which is capable of holding liquid samples S. Note that the sample holding portion 13 may not be provided, in cases that a slight amounts of liquid samples S, which can be held on the sensor chip 10 by surface tension, are to be measured.

The excitation light emitting optical system 20 is equipped with: a light source 21, constituted by a semiconductor laser (LD) or the like, for outputting the excitation light beam Lo; and a prism 22, which is provided such that one of the surfaces thereof is in contact with the dielectric plate 11. The prism 22 guides the excitation light beam Lo into the dielectric plate 11 such that the excitation light beam Lo is totally reflected at the interface between the dielectric plate 11 and the metal layer 12. Note that the prism 22 and the dielectric plate 11 are in contact via refractive index matching oil. The light source 21 is positioned such that the excitation light beam Lo enters the interface at an angle greater than or equal to a total reflection angle, through another surface of the prism 22. Further, light guiding members may be provided between the light source 21 and the prism 22 as necessary. Note that the excitation light beam Lo enters the interface as p polarized light, in order to effectively induce surface plasmon.

Known photodetectors, such as CCD's, PD's (photodiodes), photomultiplier tubes, and c-MOS's may be employed as the photodetector 30.

The housing portion 19 is configured such that when the sensor chip 10 is housed therein, the sensor portion 14 is positioned on the prism 22 and fluorescence can be detected by the photodetector 30. The sensor chip 10 is inserted into and removed from the housing portion 19 in the direction indicated by arrow X in FIG. 1.

The pH control means controls the pH of samples by dispensing/replacing/cleansing the samples with a phosphate buffer (pH=4.5), for example. The phosphate buffer is stored in a port separate from the sensor chip 10, and is injected onto the sensor portion 14 by a device equipped with a pump, dispenser, and the like. Alternatively, the buffer may be caused to flow onto the sensor portion 14 with a finger. The intensity of the charges imparted onto the sensor portion 14 and the fluorescent substance F can be controlled by varying the pH of the liquid sample with the pH control means. That is, as illustrated in FIG. 2A and FIG. 2B, the acidic functional groups and the basic functional groups receive and give protons (H⁺) depending on the surrounding pH. Accordingly, it becomes possible to control the interaction between the sensor portion 14 and the fluorescent substance F by controlling the pH. For example, in the case that the pH is varied from 7.0 as illustrated in FIG. 2A to 4.5 as illustrated in FIG. 2B, the degree of disassociation of the amino groups increases, but the degree of disassociation of the carboxyl groups decreases, and thereby the interactions between the sensor portion 14 and the fluorescent substance F can be weakened. If this property is utilized to adjust the pH thereby weakening the interactions between the sensor portion 14 and the fluorescent substance F, then the fluorescent substance F which is non specifically adsorbed is removed, then the fluorescent substance F that may lead to noise can be efficiently removed.

Hereinafter, the detecting method and the operation of the detecting apparatus according to the first embodiment will be described.

A sensor chip 10 having a positively charged metal film 12 and primary antibodies B₁ (first binding substance) that specifically bind with antigens A immobilized onto the metal film 12 is prepared. Note that the surface of the metal film is modified by a self assembling film that has amino groups in order to have positive electric charges imparted thereon (refer to FIG. 2A).

First, a sample S which is a target of inspection, is caused to flow into the sample holding portion 13, and placed in contact with the metal film 12 of the sensor chip 10. Similarly, a solution that contains a fluorescent label binding substance constituted by the negatively charged fluorescent substance F and secondary antibodies B₂ (second binding substance) that specifically bind to the antigens A, is caused to flow within the sample holding section 13. In this case, the primary antibodies B₁ that the surface of the metal film 12 is modified with and the secondary antibodies B₂ of the fluorescent label binding substance B_{F} are selected such that they bind with different epitopes of the antigen A (detection target substance). Note that the fluorescent substance F, in which the fluorescent pigment molecules f are enveloped and the surfaces of which have carboxyl groups, are employed as the fluorescent labels F (refer to FIG. 2A). In the case that the antigens A are present within the sample S, the primary antibodies B₁ will specifically bind with the antigens A, and further, the secondary antibodies B₂ of the fluorescent label binding substance B_{F} will bind with the antigens A, to form sandwich formations. Thereafter, the buffer solution is caused to flow through the sample holding portion 13 to wash away the fluorescent label binding substance B_{F} which has not undergone binding reactions.

Note that the above steps may be performed prior to setting the sensor chip 10 in the housing portion 19 of the detecting apparatus 1, or after the sensor chip 10 is set in the housing portion 19. In addition, the timing at which the detection target substance (the antigens A) is labeled is not particularly limited. Fluorescent labels (the fluorescent substance F) may be added to the sample S prior to causing the detection target substance (the antigens A) to bind with the first binding substance (the primary antibodies B₁).

In the first embodiment, the sensor portion 14 has a first electric charge on the surface thereof, and the fluorescent substance F has second electric charges on the surfaces thereof. Accordingly, the fluorescent substance F is drawn to the vicinity of the surface of the sensor portion 14 due to static electric interactions. The excitation light beam Lo is irradiated by the excitation light irradiating optical system 20 toward the predetermined region of the dielectric plate 11 of the sensor chip 10 in a state in which the fluorescent substance F is drawn toward the sensor portion 14. Evanescent light leaks into the sample S on the metal film 12, by the excitation light beam Lo entering the interface between the dielectric plate 11 and the metal film 12 at a specific incident angle greater than or equal to a total reflection angle. Surface plasmon is excited within the metal film 12 by the evanescent light. The surface plasmon forms an enhanced optical field D on the surface of the metal film 12. At this time, the fluorescent pigment molecules f within the fluorescent substance F are excited by the enhanced optical field D, and fluorescence Lf is generated. The fluorescence Lf is enhanced by the effect of the electric field enhancing effect of surface plasmon. The presence and/or the amount of the detection target substance A, which is bound to the fluorescent label binding substance B_{F}, is detected by detecting the fluorescence Lf with the photodetector 30.

As described above, in the detecting method and the detecting apparatus of the present invention, detection is performed in a state in which a first electric charge is imparted to the sensor portion, and a second electric charge opposite the first electric charge is imparted onto the fluorescent substance that functions as fluorescent labels. Accordingly, the fluorescent labels are drawn to the vicinity of the surface of the sensor portion, at which the electric field enhancing effect is great, due to static electric interactions. Therefore, amounts of light which are generated due to excitation of the fluorescent labels in a state that the fluorescent labels are in the vicinity of the surface of the sensor portion can be detected. As a result, the electric field at the surface of the sensor portion, at which the degree of enhancement is great, can be efficiently utilized, and the distances from the surface of the sensor portion to the fluorescent labels can be uniformized. Accordingly, fluctuations in signal intensities can be suppressed. That is, stable signals having favorable S/N ratios can be detected, and the presence and/or the amounts of detection target substances can be accurately detected.

Note that in the case that the metal layer 12 is present on the surface of the sensor portion 14 as in the first embodiment, the influence of metallic quenching due to the internal metal layer becomes conspicuous. Therefore, it is necessary to finely control the distances between the internal metal layer and the fluorescent pigment molecules. The degree of metal quenching is inversely proportionate to the distance between the molecules and the metal to the third power in the case that the metal is a plane which is infinitely thick. The degree of metal quenching is inversely proportionate to the distance between the molecules and the metal to the fourth power in the case that the metal is a plane which is infinitely thin. The degree of metal quenching is inversely proportionate to the distance between the molecules and the metal to the sixth power in the case that the metal is in the form of fine particles. Accordingly, it is desirable for a distance of several nm or greater, preferably 10 nm or greater, to be secured between the internal metal film and the fluorescent pigment molecules f. Control of distances in this manner is generally performed by providing a barrier layer, such as a polymer film, an SiO₂ film, an SAM film and a CMD film on the metal layer. However, the provision of such a barrier layer is troublesome and not suited for practical use. However, in the case that the fluorescent substance of the present invention includes a sufficient number of fluorescent pigment molecules, a certain amount of distance can be secured between the internal metal film and many of the fluorescent pigment molecules, without providing a film for preventing metallic quenching. Thereby, the trouble of providing the CMD film and the SAM film, which had conventionally been necessary to prevent metallic quenching, is obviated, metallic quenching can be effectively prevented by an extremely simple method, and fluorescent signals can be stably detected.

### Design Modifications to the First Embodiment

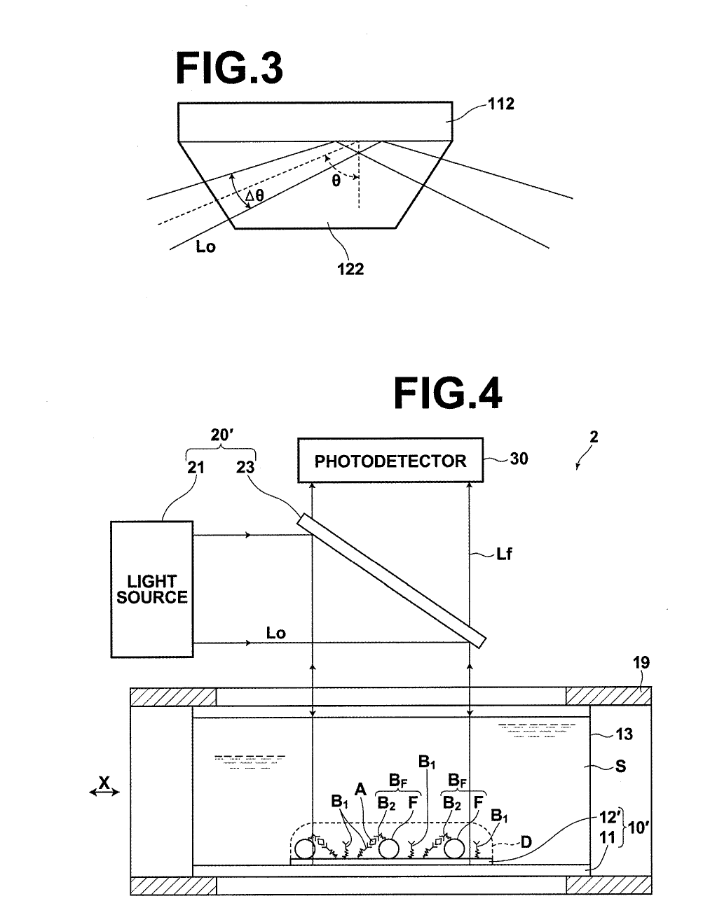

In the first embodiment described above, a parallel light beam that enters the interface at a specific angle θ was employed as the excitation light beam Lo. Alternatively, a fan beam (convergence light beam) having an angular width of Δθ with an angle θ at its center may be employed, as schematically illustrated in FIG. 3. In the case that the fan beam is employed, the fan beam enters the interface between a prism 122 and a metal layer 112a on the prism at incident angles within a range from θ-Δθ/2 to θ+Δθ/2. If a resonance angle exists within this angular range, second evanescent waves can be induced on an optical waveguide layer. The refractive index of media on optical waveguide layers changes before and after supply of a sample thereon, and therefore, the resonance angle at which the optical waveguide mode is induced changes. For this reason, in the case that a parallel light beam is employed as the excitation light beam Lo as in the embodiments described above, it becomes necessary to adjust the incident angle of the parallel light beam each time that the resonance angle changes. However, the changes in resonance angles can be dealt with without adjusting the incident angle, by employing the fan beam having the width in incident angles that enter the interface as illustrated in FIG. 3. Note that it is preferable for the fan beam to have a flat distribution with little variations in intensity corresponding to the incident angles. The same applies to the following second through fifth embodiments.

### Second Embodiment

A detecting method and a detecting apparatus according to a second embodiment will be described with reference to FIG. 4. FIG. 4 is a schematic diagram that illustrates the entirety of the detecting apparatus of the second embodiment. The detecting method and the detecting apparatus of the second embodiment enhances a optical field by localized plasmon resonance, and detects fluorescence which is excited by the enhanced optical field. Note that in the following description, components which are the same as those of the first embodiment are denoted with the same reference numerals.

A sensor chip 10′ and an excitation light irradiating optical system 20′ of the fluorescence detecting apparatus 2 illustrated in FIG. 4 differs from the fluorescence detecting apparatus 1 of the first embodiment.

The sensor chip 10′ is equipped with a finely structured metal piece having recesses and protrusions at a frequency smaller than the wavelength of the excitation light beam Lo, or a plurality of metal nano rods of sizes smaller than the wavelength of the excitation light beam Lo as a metal layer 12′. In the case that the sensor chip 10′ is equipped with the meta layer 12′ that causes localized plasmon to be generated, it is not necessary to cause the excitation light beam Lo to enter the interface between the metal layer 12′ and the dielectric plate 11 such that conditions for total reflection are satisfied. Accordingly, the excitation light irradiating optical system 20′ is configured to irradiate the excitation light beam Lo from above the dielectric plate 11.

The excitation light irradiating optical system 20′ is equipped with a light source 21 constituted by a semiconductor layer (LD) or the like for outputting the excitation light beam Lo, and a half mirror 23 for reflecting and guiding the excitation light beam Lo toward the sensor chip 10′. The half mirror 23 reflects the excitation light beam Lo, and transmits fluorescence Lf.

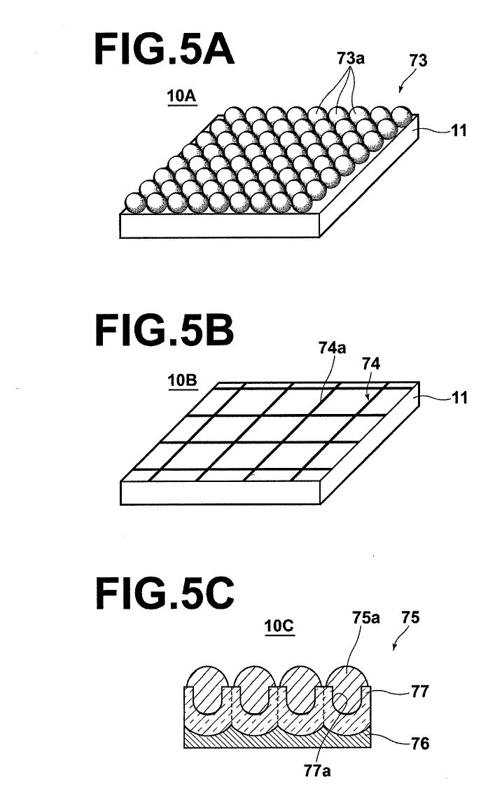

Specific examples of the sensor chip 10′ will be described with reference to FIGS. 5A through 5C.

Sensor chip 10A illustrated in FIG. 5A is constituted by a dielectric plate 11, and a finely structured metal piece 73 formed by a plurality of metal particles 73a, which are fixed onto a predetermined region of the dielectric plate 11 in the form of an array. The arrangement pattern of the metal particles 73a may be designed as appropriate, but it is preferable for the arrangement pattern to be substantially regular. In this structure, the average size and the pitch of the metal particles 73a are smaller than the wavelength of the excitation light beam Lo.

Sensor chip 10B illustrated in FIG. 5B is constituted by a dielectric plate 11, and a finely structured metal piece 74 formed by a metal pattern layer, in which thin metal wires 74a are patterned in a lattice. The pattern of the metal pattern layer may be designed as appropriate, but it is preferable for the pattern to be substantially regular. In this structure, the average thickness of the thin metal wires 74a and the pitch thereof are smaller than the wavelength of the excitation light beam Lo.

Sensor chip 10C illustrated in FIG. 5C is constituted by a finely structured metal piece 75 formed by a metal piece 76, a metal oxide layer having a plurality of fine apertures 77a, and a plurality of mushroom shaped metal pieces 75a which are formed within the fine apertures 77a. The metal piece 76 and the metal oxide layer having the plurality of fine apertures 77a may be formed by anodizing a portion of a metal piece formed of Al or the like, as described in U.S. Patent Application Publication No. 20070158549, to obtain a metal oxide piece (such as Al₂O₃). Here, the metal oxide piece 77 corresponds to the dielectric plate. The finely structured metal piece 75 is obtained by growing the metal pieces 75a in each of the fine apertures 77a by plating or the like.

In the example illustrated in FIG. 5C, the head portions of the mushroom shaped metal pieces 75a are particle shaped, and the structure is an arrangement of fine metal particles, if viewed from the surface of a sample plate. In this structure, the head portions of the mushroom shaped metal pieces 75a are protrusions, and the average size and the pitch thereof are smaller than the wavelength of the excitation light beam Lo.

Note that other examples of the metal layer 12′ at which localized plasmon is generated by irradiation of excitation light include fin structured pieces obtained by anodizing metal, as described in U.S. Patent Application Publication Nos. 20060234396 and 20060181701.

Further, the metal layer at which localized plasmon is generated may be constituted by a metal film of which the surface has been roughened. Examples of a roughening method include an electrochemical method that utilizes oxidation reduction or the like. As another alternative, the metal layer may be constituted by a plurality of metal nano rods which hare arranged on a sample plate. The dimension of the short axis of the nano rods may be within a range from 3 nm to 50 nm, and the dimension of the long axis of the nano rods may be within a range from 25 nm to 1000 nm. The size of the nano rods in the long axis is set to be smaller than the wavelength of the excitation light beam Lo. Metal nano rods are described in U.S. Patent Application Publication No. 20070118936, for example.

Note that it is preferable for the material of the finely structured metal piece or the metal nano rods which are employed as the metal layer 12′ to be a metal having at least one of Au, Ag, Cu, Al, Pt, Ni, Ti, and alloys thereof as a main component.

The fluorescence detecting method that employs the fluorescence detecting apparatus 2 of the second embodiment will be described.

The steps of preparing the sensor chip and causing the antigen antibody reaction are the same as those of the first embodiment, and therefore, a detailed description thereof will be omitted here. The same applies to the following embodiments as well.

In the second embodiment, the sensor portion 14 has a first electric charge on the surface thereof, and the fluorescent substance F has second electric charges on the surfaces thereof. Accordingly, the fluorescent substance F is drawn to the vicinity of the surface of the sensor portion 14 due to static electric interactions. The excitation light beam Lo is irradiated by the excitation light irradiating optical system 20 toward the predetermined region of the dielectric plate 11 of the sensor chip 10′ in a state in which the fluorescent substance F is drawn toward the sensor portion 14. The excitation light beam Lo emitted by the light source 21 is reflected by the half mirror 23 and enters the sample contacting surface of the sensor chip 10′. Localized plasmon is excited at the surface of the metal layer 12′ due to irradiation of the excitation light beam Lo. The localized plasmon forms an enhanced optical field D on the surface of the metal film 12. At this time, the fluorescent pigment molecules f within the fluorescent substance F are excited by the enhanced optical field D, and fluorescence Lf is generated. The presence and/or the amount of the detection target substance A, which is bound to the fluorescent label binding substance B_{F}, is detected by detecting the fluorescence Lf with the photodetector 30.

In the second embodiment as well, the first electric charge is imparted to the surface of the sensor portion 14, and the second electric charge is imparted onto the surfaces of the fluorescent substance F. Therefore, the fluorescence Lf is detected in a state in which the fluorescent substance F is drawn to the vicinity of the surface of the sensor portion 14. Accordingly, the same advantageous effects as those obtained by the first embodiment can be obtained.

### Third Embodiment

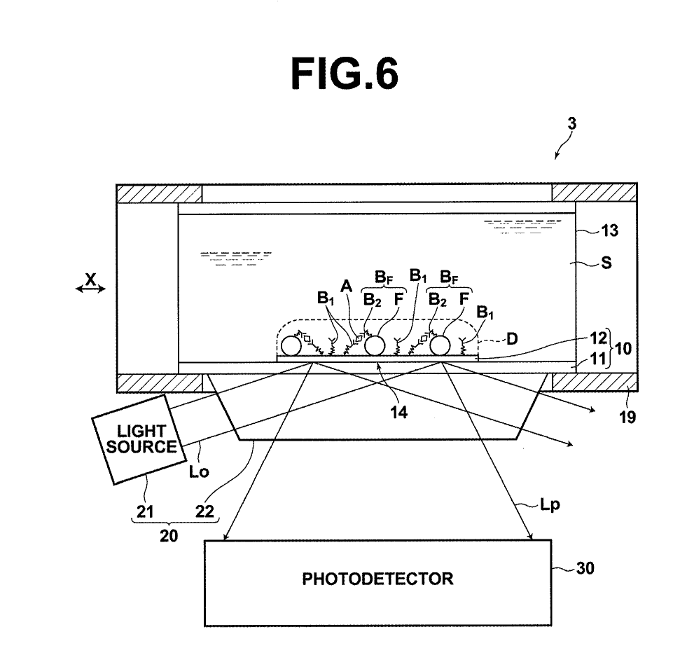

A detecting method and a detecting apparatus according to a third embodiment will be described with reference to FIG. 6. FIG. 6 is a schematic diagram that illustrates the entirety of the detecting apparatus of the third embodiment. The detecting method and the detecting apparatus of the third embodiment enhances a optical field by surface plasmon resonance, and detects radiant light that radiates toward the side of a dielectric plate opposite the side on which a metal layer is formed, from surface plasmon which is newly excited at the metal layer by fluorescence generated by fluorescent labels due to excitation.

The radiant light detecting apparatus 3 illustrated in FIG. 6 differs from the fluorescence detecting apparatus of the first embodiment in the placement of the photodetector 30. The photodetector 30 is positioned so as to detect radiant light Lp, which is radiated toward the side of the dielectric plate opposite the side on which a metal layer is formed, from surface plasmon which is newly excited at the metal layer by fluorescence generated by the fluorescent labels due to excitation.

The radiant light detecting method of the third embodiment that employs the detecting apparatus 3 will be described.

In the third embodiment, the sensor portion 14 has a first electric charge on the surface thereof, and the fluorescent substance F has second electric charges on the surfaces thereof. Accordingly, the fluorescent substance F is drawn to the vicinity of the surface of the sensor portion 14 due to static electric interactions. The excitation light beam Lo is irradiated by the excitation light irradiating optical system 20 toward the interface between the dielectric plate 11 and the metal film 12 in a state in which the fluorescent substance F is drawn toward the sensor portion 14, as in the first embodiment. Evanescent light leaks into the sample S on the metal film 12, by the excitation light beam Lo entering the interface between the dielectric plate 11 and the metal film 12 at a specific incident angle greater than or equal to a total reflection angle. Surface plasmon is excited within the metal film 12 by the evanescent light. The surface plasmon forms an enhanced optical field D on the surface of the metal film 12. At this time, the fluorescent pigment molecules f within the fluorescent substance F are excited by the enhanced optical field D, and fluorescence Lf is generated. The fluorescence Lf is enhanced by the effect of the electric field enhancing effect of surface plasmon. The fluorescence Lf which is generated on the metal film 12 newly induces surface plasmon on the surface of the metal film 12, and radiant light Lp is emitted at a specific angle toward the side of the sensor chip 10 opposite the surface on which the metal film 12 is formed. The presence and/or the amount of the detection target substance A, which is bound to the fluorescent label binding substance B_{F}, is detected by detecting the radiant light Lp with the photodetector 30.

The radiant light Lp is generated when the fluorescence Lf couples with surface plasmon of a specific wave number at the metal layer 12. The wavelength of the fluorescence Lf determines the wave number at which the coupling with the surface plasmon occurs. Therefore, the radiant angle of the radiant light Lp is determined according to the wave number. Generally, the wavelength of the excitation light beam Lo and the wavelength of the fluorescence Lf are different. Therefore, wave number of the surface plasmon which is excited by the fluorescence Lf is different from the wave number of the surface plasmon which is excited by the excitation light beam Lo, and accordingly, the radiant light Lp is radiated at an angle different from the incident angle of the excitation light beam Lo.

In the third embodiment as well, the first electric charge is imparted to the surface of the sensor portion 14, and the second electric charge is imparted onto the surfaces of the fluorescent substance F. Therefore, the fluorescence Lf is generated in a state in which the fluorescent substance F is drawn to the vicinity of the surface of the sensor portion 14, and the radiant light Lp generated due to the enhanced fluorescence Lf is detected. Accordingly, the same advantageous effects as those obtained by the first embodiment can be obtained.

Further, the third embodiment detects the light generated due to fluorescence generated at the surface of the sensor from the rear side of the sensor. Therefore, the distance that the fluorescence Lf travels through media that absorbs light can be reduced to several 10's of nanometers. Accordingly, light absorption by blood, for example, becomes negligible, and measurement becomes possible without performing preliminary processes of removing coloring components such as red blood cells from blood with a centrifuge, and passing blood through blood cell filters to obtain blood serum or plasma.

### Fourth Embodiment

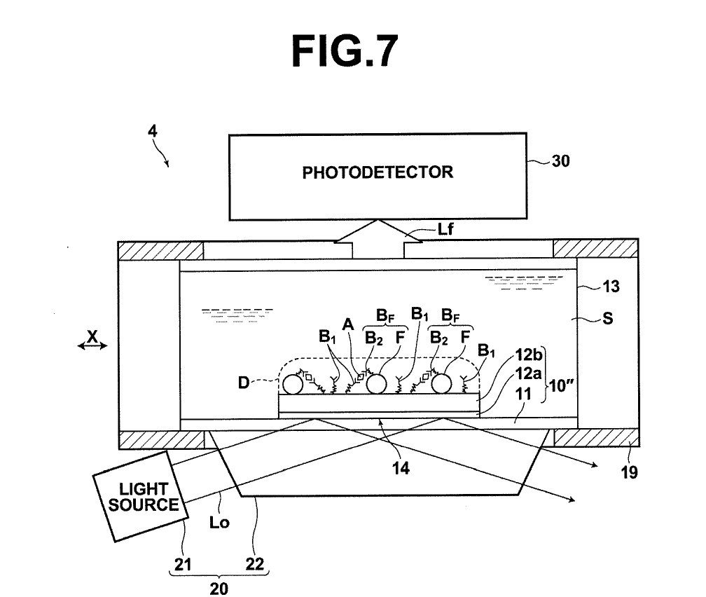

A detecting method and a detecting apparatus according to a fourth embodiment will be described with reference to FIG. 7. FIG. 7 is a schematic diagram that illustrates the entirety of the detecting apparatus 4 of the fourth embodiment. The detecting method and the detecting apparatus of the fourth embodiment employs a sensor chip equipped with an optical waveguide layer on a metal layer. An optical waveguide mode is excited at the optical waveguide layer, which generates an enhanced optical field. Fluorescence which is excited by the enhanced optical field is detected.

The construction of the detecting apparatus 4 illustrated in FIG. 7 is the same as the construction of the detecting apparatus of the first embodiment. However, the sensor chip which is employed is different, and the different sensor chip results in a different mechanism of electric field enhancement.

A sensor chip 10″ which is utilized in the fourth embodiment is equipped with a metal layer 12a and an optical waveguide layer 12b on the metal layer 12a. The thickness of the optical waveguide layer 12b is not particularly limited, and may be determined such that the optical waveguide mode is induced, taking the wavelength and incident angle of the excitation light beam Lo and the refractive index of the optical waveguide layer 12b into consideration. For example, in the case that a laser beam having a central wavelength of 780 nm is employed as the excitation light beam Lo and a silicon oxide film is employed as the optical waveguide layer 12b, it is preferable for the thickness of the optical waveguide layer 12b to be within a range from 500 nm to 600 nm. The optical waveguide layer 12b may be of a laminated structure that includes at least one internal optical waveguide layer constituted by optical waveguiding material. It is preferable for the laminated structure to be of an alternating laminated structure, in which the internal optical wave guide layer and an internal metal layer are alternately provided in this order from the side of the metal layer 12a.

The fluorescence detecting method of the fourth embodiment that employs the detecting apparatus 4 will be described.

In the fourth embodiment, the sensor portion 14 has a first electric charge on the surface thereof, and the fluorescent substance F has second electric charges on the surfaces thereof. Accordingly, the fluorescent substance F is drawn to the vicinity of the surface of the sensor portion 14 due to static electric interactions. The excitation light beam Lo is irradiated by the excitation light irradiating optical system 20 toward the interface between the dielectric plate 11 and the metal layer 12a in a state in which the fluorescent substance F is drawn toward the sensor portion 14, as in the first embodiment. Evanescent light leaks onto the metal layer 12a, by the excitation light beam Lo entering the interface between the dielectric plate 11 and the metal layer 12a at a specific incident angle greater than or equal to a total reflection angle. The evanescent light couples with an optical waveguide mode of the optical waveguide layer 12b, to excite an optical waveguide mode. The optical waveguide mode forms an enhanced optical field D on the optical waveguide layer 12b. At this time, the fluorescent pigment molecules f within the fluorescent substance F are excited by the enhanced optical field D, and fluorescence Lf is generated. The fluorescence Lf is enhanced by the effect of the electric field enhancing effect of the optical waveguide mode. The presence and/or the amount of the detection target substance A, which is bound to the fluorescent label binding substance B_{F}, is detected by detecting the fluorescence Lf with the photodetector 30.

In the fourth embodiment as well, the first electric charge is imparted to the surface of the sensor portion 14, and the second electric charge is imparted onto the surfaces of the fluorescent substance F. Therefore, the fluorescence Lf is detected in a state in which the fluorescent substance F is drawn to the vicinity of the surface of the sensor portion 14. Accordingly, the same advantageous effects as those obtained by the first embodiment can be obtained.

Further, the optical field D which is enhanced by the optical waveguide mode attenuates more gradually along with distances from the surface of the sensor portion than optical field which are enhanced by surface plasmon. Therefore, in the case that the fluorescent substance F which is employed has a plurality of fluorescent pigment molecules enveloped therein and has large diameters, a greater fluorescent intensity enhancing effect can be obtained compared to fluorometry that employs surface plasmon to enhance electric fields.

### Fifth Embodiment

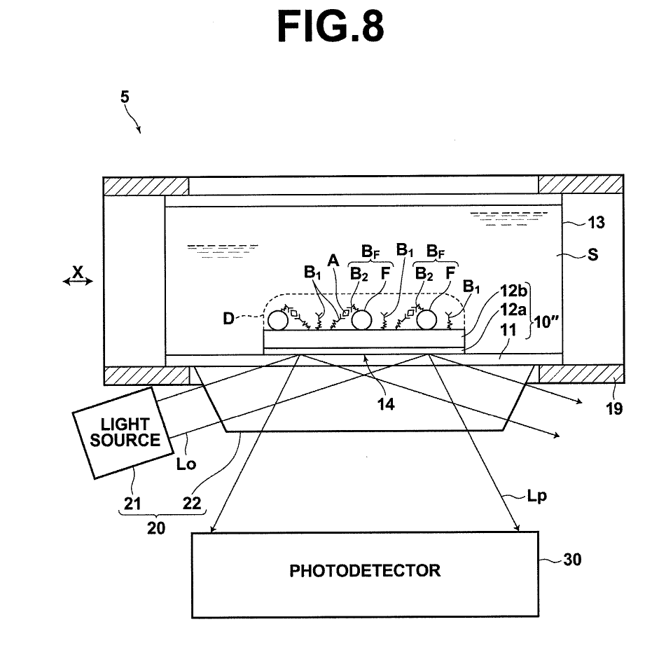

A detecting method and a detecting apparatus according to a fifth embodiment will be described with reference to FIG. 8. FIG. 8 is a schematic diagram that illustrates the entirety of the detecting apparatus 5 of the fifth embodiment. The detecting method and the detecting apparatus of the fifth embodiment employs a sensor chip equipped with an optical waveguide layer on a metal layer. An optical waveguide mode is excited at the optical waveguide layer, which generates an enhanced optical field. Radiant light that radiates toward the side of a dielectric plate opposite the side on which a metal layer is formed, from surface plasmon which is newly excited at the metal layer by fluorescence generated by fluorescent labels due to excitation is detected.

The construction of the detecting apparatus 5 illustrated in FIG. 8 is the same as the construction of the detecting apparatus of the third embodiment. The sensor chip which is employed in the detecting method of the fifth embodiment is the same as the sensor chip which is employed in the detecting method of the fourth embodiment.

The radiant light detecting method of the fifth embodiment that employs the detecting apparatus 5 will be described.

In the fifth embodiment, the sensor portion 14 has a first electric charge on the surface thereof, and the fluorescent substance F has second electric charges on the surfaces thereof. Accordingly, the fluorescent substance F is drawn to the vicinity of the surface of the sensor portion 14 due to static electric interactions. The excitation light beam Lo is irradiated by the excitation light irradiating optical system 20 toward the interface between the dielectric plate 11 and the metal layer 12a in a state in which the fluorescent substance F is drawn toward the sensor portion 14, as in the first embodiment. Evanescent light leaks onto the metal layer 12a, by the excitation light beam Lo entering the interface between the dielectric plate 11 and the metal layer 12a at a specific incident angle greater than or equal to a total reflection angle. The evanescent light couples with an optical waveguide mode of the optical waveguide layer 12b, to excite an optical waveguide mode. The optical waveguide mode forms an enhanced optical field D on the optical waveguide layer 12b. At this time, the fluorescent pigment molecules f within the fluorescent substance F are excited by the enhanced optical field D, and fluorescence Lf is generated. The fluorescence Lf is enhanced by the effect of the electric field enhancing effect of the optical waveguide mode. The fluorescence Lf which is generated on the metal film 12 newly induces surface plasmon on the surface of the metal film 12, and radiant light Lp is emitted at a specific angle toward the side of the sensor chip 10 opposite the surface on which the metal film 12 is formed. The presence and/or the amount of the detection target substance A, which is bound to the fluorescent label binding substance B_{F}, is detected by detecting the radiant light Lp with the photodetector 30.

In the fifth embodiment as well, the first electric charge is imparted to the surface of the sensor portion 14, and the second electric charge is imparted onto the surfaces of the fluorescent substance F. Therefore, the fluorescence Lf is generated in a state in which the fluorescent substance F is drawn to the vicinity of the surface of the sensor portion 14, and the radiant light Lp generated due to the enhanced fluorescence Lf is detected. Accordingly, the same advantageous effects as those obtained by the first embodiment can be obtained.

In addition, the fifth embodiment detects the light generated due to fluorescence generated at the surface of the sensor from the rear side of the sensor. Therefore, the distance that the fluorescence Lf travels through media that absorbs light can be reduced to several 10's of nanometers. Accordingly, the same advantageous effects as those obtained by the third embodiment can be obtained.

Further, because the optical field which is enhanced by the optical waveguide mode is employed, a fluorescent intensity increasing effect can be obtained in the same manner as in the fourth embodiment.

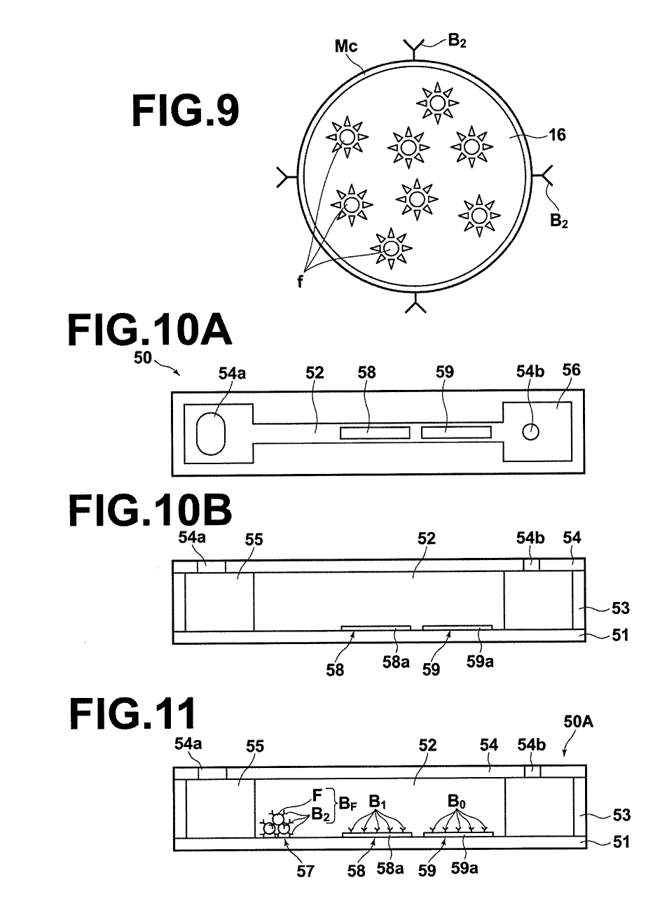

Note that in the first through fifth embodiments described above, metal coating films Mc of thicknesses that transmit fluorescence may be provided on the surfaces of the light transmitting material 16 of the fluorescent labels, as illustrated in FIG. 9. The metal coating films Mc may cover the entireties of the surfaces of the light transmitting material 16, or may partially cover the surfaces of the light transmitting material 16 such that the light transmitting material 16 is exposed at portions. The same materials which are employed for the metal layer may be employed as the material of the metal coating films Mc. In the case that the metal coating films Mc are provided, the metal coating films Mc may be modified with thiol groups, and a self assembling film having different functional groups may be modified by new functional groups.

In the case that the metal coating films Mc are provided on the surfaces of the fluorescent substance F, the surface plasmon, the localized plasmon, or the optical waveguide mode generated on the metal layer 12, the metal layer 12′, or the optical waveguide layer 12b of the sensor chip 10, the sensor chip 10′, or the sensor chip 10″ couples with the whispering gallery mode of the metal coating films Mc, and the fluorescent pigment molecules f within the fluorescent substance F can be excited at higher efficiency. Note that the whispering gallery mode is an electromagnetic wave mode which is locally present on the surfaces of fine spheres such as the fluorescent substance F having a particle diameter less than or equal to φ5300 nm, and revolves about the periphery of the spheres.

An example of a method for coating the fluorescent substance with the metal coating film will be described.

First, the fluorescent substance is produced by the steps described previously, and the surfaces thereof are modified with PEI (Poly Ethyl Imine).

Next, Pd nano particles having particle sizes of 15 nm (by Tokuriki, average particle size of 19 nm) are adsorbed onto the PEI on the surfaces of the fluorescent substance.

The polystyrene particles, onto which the PD nano particles are adsorbed, are immersed in an immersion gold plating liquid (HAuCl₄ by Kojima Chemical). Thereby, immersion plating that employs the Pd nano particles as catalysts is utilized to form gold films on the surfaces of the polystyrene particles.

### [Detection Sample Cell]

Detection sample cells which are utilized as the sensor chips in the detecting methods of the present invention will be described.

### First Embodiment

FIG. 10A is a plan view that illustrates the construction of a detection sample cell 50, and FIG. 10B is a cross sectional side view of the detection sample cell 50.

The detection sample cell 50 is equipped with: a base 51, which is formed by a dielectric plate; a spacer 53 for holding the liquid sample S on the base 51 and which forms a channel 52 for the liquid sample S; and an upper plate 54, which is a glass plate having an injection opening 54a through which the liquid sample S is injected, and an air aperture 54b through which the liquid sample S is expelled after flowing through the channel 52. Metal layers 58a and 59a are provided at predetermined regions of the base 51 between the injection opening 54a and the air aperture 54b, that is, at sample contacting surfaces of the base 51, to form sensor portions 58 and 59. Here, the sensor portions have first electrical charges imparted on the surfaces thereof. A membrane filter 55 is provided at a position between the injection opening 54a and the channel 52, and a waste liquid repository is formed at the downstream portion of the channel 52 where the channel 52 connects with the air aperture 54b.

In the detection sample cell of the first embodiment, the base 51 is a dielectric plate, and functions as the dielectric plate of the sensor portions. Alternatively, only the portions of the base 51 that become the sensor portions may be formed by dielectric plates. The detection sample cell 50 may be utilized as the sensor chip in the detecting methods and the detecting apparatuses according to the first through third embodiments described above. The detection sample cell 50 may be utilized by immobilizing a first binding substance that specifically binds with a detection target substance onto the sensor portions. Further, the detection sample cell 50 may also be equipped with: one of a second binding substance that specifically binds with the detection target substance and a third binding substance that specifically binds with the first binding substance and competes with the detection target substance; and a fluorescent label binding substance modified with one of the second binding substance and third binding substance, and on the surfaces of which second electric charges opposite the electric charges of the sensor portion are imparted, provided in the channel upstream from the sensor portions.

The detection sample cell of the first embodiment is equipped with the sensor chip having the first electric charges on the surfaces of the sensor portions thereof, and the fluorescent substance F having fluorescent pigment molecules f enveloped therein and the second charges on the surfaces thereof. Accordingly, the amount of light which is generated due to excitation of the fluorescent labels can be easily detected in a state in which the fluorescent labels are drawn to the vicinities of the surfaces of the sensor portions where the electric field enhancing effect is great, due to static electric interactions. As a result, the electric field at the surface of the sensor portion, at which the degree of enhancement is great, can be efficiently utilized, and the distances from the surface of the sensor portion to the fluorescent labels can be uniformized. Accordingly, fluctuations in signal intensities can be suppressed. That is, stable signals having favorable S/N ratios can be detected, and the presence and/or the amounts of detection target substances can be accurately detected.

### Second Embodiment

FIG. 11 is a side view of a detection sample cell 50A according to a second embodiment of the present invention, which is suited for performing assays by the sandwich method. In the detection sample cell 50A, a labeling secondary antibody adsorption area 57, the first sensor portion 58, and the second sensor portion 59 are provided in this order on the base 51 from the upstream side of the channel 52. Labeling secondary antibodies B_{F} (fluorescent label biding substance) constituted by the fluorescent substance F, the surfaces of which are modified with secondary antibodies B₂ (second binding substance) that specifically bind to antigens to be detected, are physically adsorbed onto the labeling secondary antibody adsorption area. Primary antibodies B₁ (first binding substance) that specifically bind with the antigens to be detected are immobilized onto the first sensor portion 58. Primary antibodies B₀ that do not specifically bind to the antigen to be detected but specifically bind to the labeling secondary antibodies B₂ are immobilized onto the second sensor portion 59. In the present example, two sensor portions are provided. Alternatively, a single sensor portion may be provided. The surfaces of the first sensor portion 58 and the second sensor portion 59 are charged with first electric charges. The surfaces of the fluorescent label binding substance B_{F} are charged with second electric charges opposite the first electric charges.

Au films 58a and 59a are provided as the metal layer on the base 51 at the first sensor portion 58 and the second sensor portion 59, respectively. Primary antibodies B₁ are immobilized on the Au film 58a at the first sensor portion 58. Primary antibodies B₀, which are different from the primary antibodies B₁, are immobilized on the Au film 59a at the second sensor portion 59. Self assembling films that have functional groups to impart the first electric charges are also immobilized onto the Au film 58a and the Au film 59a. The first sensor portion 58 and the second sensor portion 59 are of the same construction, except that different primary antibodies are immobilized thereon. The primary antibodies B₀ which are immobilized at the second sensor portion 59 do not bind with the antigens A, but directly bind with the labeling secondary antibodies B₂. Thereby, the amount and activity of the labeling secondary antibodies which have flowed through the channel 52, which are factors of variation of binding reactions, and factors of variation due to the optical field enhancement, such as the excitation light emitting optical system 20, the Au films 58a and 59a, and the liquid sample S, can be detected and utilized for calibration. Note that a known amount of a labeling substance may be immobilized on the second sensor portion 59 instead of the primary antibodies B₀. The labeling substance may be the same as the fluorescent substance F, the surfaces of which have been modified with the secondary antibodies B₂. Alternatively, the labeling substance may be a fluorescent substance having a different wavelength and of a different size. Further, the labeling substance may be fine metallic particles. In this case, only the factors of variation due to the enhancing effect of surface plasmon, such as the excitation light emitting optical system 20, the Au films 58a and 59a, and the liquid sample S, can be detected and utilized for calibration. Whether to provide the labeling secondary antibodies B₂ or the known amount of the labeling substance at the second sensor portion 59 may be determined according to the purposes and method of calibration.

The detection sample cell 50A may be utilized as the sensor chip in the detecting methods and the detecting apparatuses according to the first through third embodiments described above. The detection sample cell 50A is configured to be movable in the X direction relative to the excitation light irradiating optical system 20 and the photodetector 30 within the housing portion 19. Thereby, after detecting and measuring fluorescence or radiant light generated at the first sensor portion 58, the second sensor portion 59 may be moved to a detecting position to detect fluorescence or radiant light generated at the second sensor portion 59.

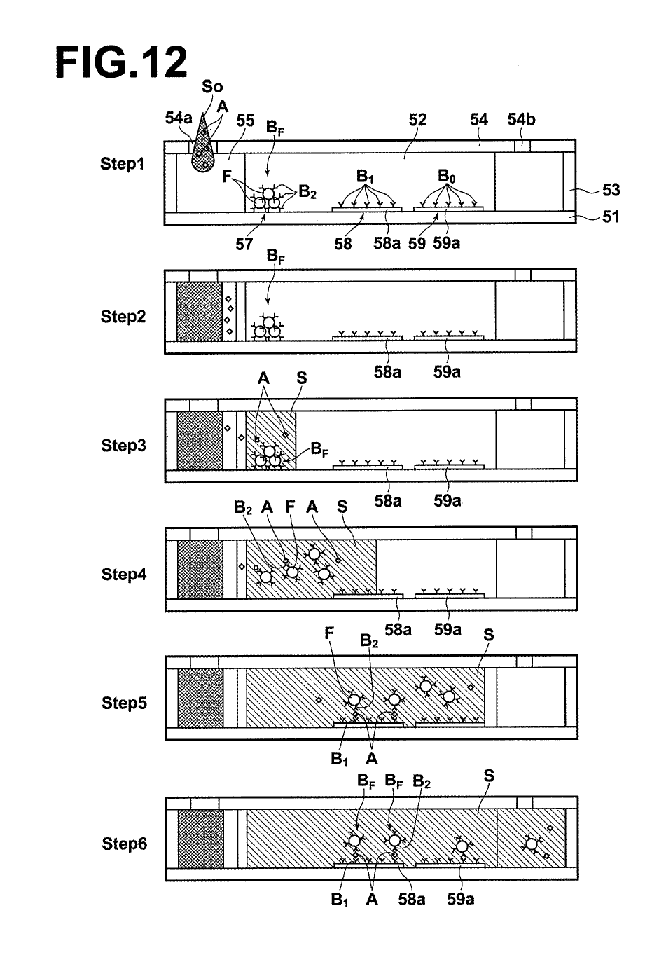

The procedures by which an assay is performed according to the sandwich method to detect whether an antigen to be detected is included in blood (whole blood) using the detection sample cell 50A of the second embodiment will be described with reference to FIG. 12.

Step 1: The blood So (whole blood), which is the target of inspection, is injected through the injection opening 54a. Here, a case will be described in which the blood So includes the antigen A to be detected. In FIG. 12, the blood So is represented by the cross hatched regions.

Step 2: The blood So is filtered by the membrane filter 55, and large molecules, such as red blood cells and white blood cells, are separated as residue.

Step 3: Plasma S (the blood from which blood cells have been filtered out by the membrane filter 55) leaks out into the channel 52 by capillary action. Alternatively, in order to expedite reactions and to shorten detection time, a pump may be connected to the air aperture 54b, and the plasma S may be caused to flow by suctioning and extruding operations of the pump. In FIG. 12, the plasma S is represented by the hatched regions.

Step 4: the plasma S, which has leaked into the channel 52 and the labeling secondary antibodies B_{F}, which are provided within the channel 52, mix, and the antigens A within the plasma S bind with the labeling secondary antibodies B_{F}.

Step 5: the plasma S gradually flows along the channel 52 toward the air aperture 54b, and the antigens A, which are bonded to the labeling secondary antibodies B_{F}, bind with the primary antibodies B₁, which are immobilized onto the first sensor portion 58. So called sandwich configurations, in which the antigens A are sandwiched between the primary antibodies B₁ and the labeling secondary antibodies B_{F}, are formed.

Step 6: A portion of the labeling secondary antibodies B_{F} that did not bind with the antigens A bind with the primary antibodies B₀. Further, even in the case that the labeling secondary antibodies B_{F} which did not bind with the antigens A or the primary antibodies B₀ remain, the following plasma S functions as a cleansing agent that washes the labeling secondary antibodies B_{F}, which are floating or non specifically bound onto the plate, away.

In this manner, the blood So is injected through the injection opening 54a, and step 1 through step 6 are performed to form the sandwich configurations, in which the antigens A are sandwiched between the primary antibodies B₁ and the labeling secondary antibodies B₂, on the first sensor portion 58. Thereafter, fluorescent signals or radiant light signals (hereinafter, detection signals) are detected at the first sensor portion 58, to detect the presence and/or the concentration of the antigens. Next, the sample cell 50 is moved in the X direction so as to enable detection signal detection at the second sensor portion 59, and detection signals are detected at the second sensor portion 59. The fluorescent signals obtained at the second sensor portion 59, at which the primary antibodies B₀ that bind with the labeling secondary antibodies B_{F} are immobilized, are considered to be fluorescent signals that reflect reaction conditions such as the amount of the labeling secondary antibodies B_{F} which has flowed through the channel 52 and the activity thereof. Therefore, if the fluorescent signals obtained at the second sensor portion 59 are used as a reference to correct the fluorescent signals obtained at the first sensor portion 58, more accurate detection results can be obtained. In the case that the known amount of the labeling substance (fluorescent substance or fine metallic particles) is immobilized onto the second sensor portion 59 in advance as described previously, the fluorescent signals obtained at the second sensor portion 59 can be used as a reference to correct the fluorescent signals obtained at the first sensor portion 58 as well.

The detection sample cell 50A of the second embodiment is also equipped with the sensor chip having the sensor portions with the first electric charges on the surfaces thereof, and the fluorescent substance F having the fluorescent pigment molecules f enveloped therein and the second electric charges on the surfaces thereof. Accordingly, the same advantageous effects which are obtained by the detection sample cell of the first embodiment can also be obtained by the detection sample cell of the second embodiment.

### Third Embodiment

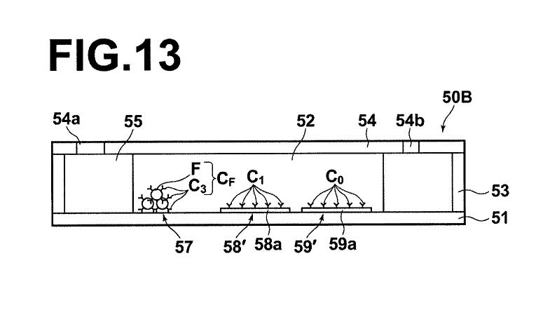

FIG. 13 is a side view of a detection sample cell 50B according to a third embodiment of the present invention, which is suited for performing assays by the competition method. In the detection sample cell 50B, a labeling secondary antibody adsorption area 57′, a first sensor portion 58′, and a second sensor portion 59′ are provided in this order on the base 51 from the upstream side of the channel 52. Labeling secondary antibodies C_{F} (fluorescent label biding substance) constituted by the fluorescent substance F, the surfaces of which are modified with Secondary antibodies C₃ (third binding substance) that do not bind with the antigens to be detected but specifically bind with primary antibodies C₁ to be described later, are physically adsorbed onto the labeling secondary antibody adsorption area 57′. Primary antibodies C₁ (first binding substance) that specifically bind with the antigens to be detected and the secondary antibodies C₃ are immobilized onto the first sensor portion 58′. Primary antibodies C₀ that do not specifically bind to the antigen to be detected but specifically bind to the labeling secondary antibodies C_{F} are immobilized onto the second sensor portion 59′. In the present example, two sensor portions are provided. Alternatively, a single sensor portion may be provided. The surfaces of the first sensor portion 58′ and the second sensor portion 59′ are charged with first electric charges. The surfaces of the fluorescent label binding substance C_{F} are charged with second electric charges opposite the first electric charges.

Gold (Au) films 58a and 59a are provided as the metal layer on the base 51 at the first sensor portion 58′ and the second sensor portion 59′, respectively. The primary antibodies C₁ are immobilized on the Au film 58a at the first sensor portion 58′. Primary antibodies C₀, which are different from the primary antibodies C₁, are immobilized on the Au film 59a at the second sensor portion 59′. Self assembling films that have functional groups to impart the first electric charges are also immobilized onto the Au film 58a and the Au film 59a. The first sensor portion 58′ and the second sensor portion 59′ are of the same construction, except that different primary antibodies are immobilized thereon. The primary antibodies C₀ which are immobilized at the second sensor portion 59 do not bind with the antigens A, but directly bind with the labeling secondary antibodies C_{F}. Thereby, the amount and activity of the labeling secondary antibodies C_{F} which have flowed through the channel 52, which are factors of variation of binding reactions, and factors of variation due to the optical field enhancement, such as the excitation light emitting optical system 20, the gold (Au) films 58a and 59a, and the liquid sample S, can be detected and utilized for calibration. Note that a known amount of a labeling substance may be immobilized on the second sensor portion 59 instead of the primary antibodies C₀. The labeling substance may be the same as the fluorescent substance F, the surfaces of which have been modified with the secondary antibodies. Alternatively, the labeling substance may be a fluorescent substance having a different wavelength and of a different size. In this case, only the factors of variation due to the enhancing effect of surface plasmon, such as the excitation light emitting optical system 20, the gold (Au) films 58a and 59a, and the liquid sample S, can be detected and utilized for calibration. Whether to provide the labeling secondary antibodies B₂ or the known amount of the labeling substance at the second sensor portion 59 may be determined according to the purposes and method of calibration.

The detection sample cell 50B may be utilized as the sensor chip in the detecting methods and the detecting apparatuses according to the first through third embodiments described above.

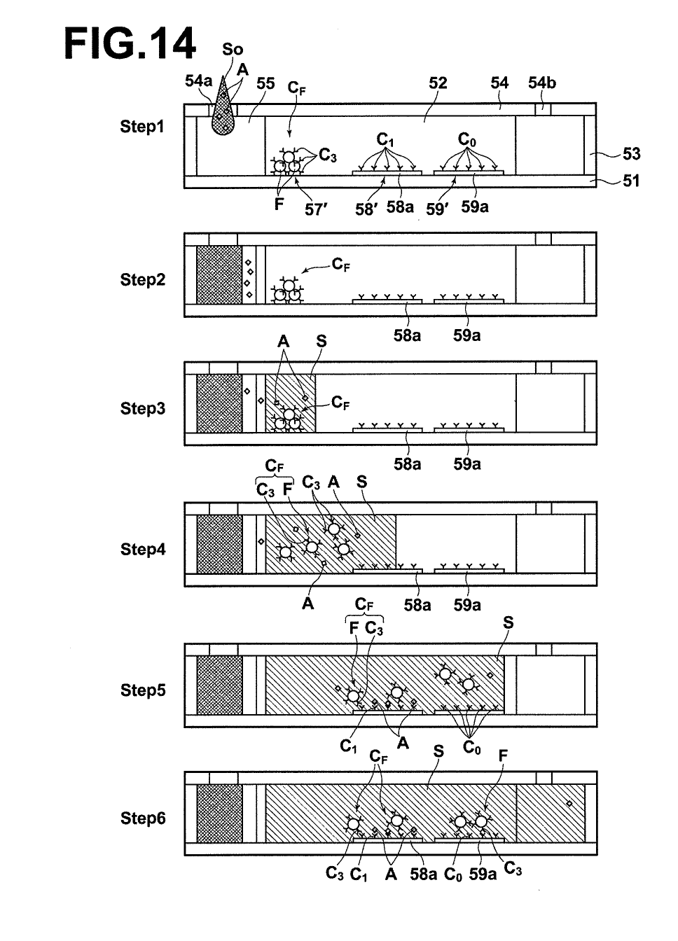

The procedures by which an assay is performed according to the competition method to detect whether an antigen to be detected is included in blood (whole blood) using the detection sample cell 50B of the third embodiment will be described with reference to FIG. 14.

Step 1: The blood So (whole blood), which is the target of inspection, is injected through the injection opening 54a. Here, a case will be described in which the blood So includes the antigen A to be detected. In FIG. 14, the blood So is represented by the cross hatched regions.

Step 2: The blood So is filtered by the membrane filter 55, and large molecules, such as red blood cells and white blood cells, are separated as residue.

Step 3: Plasma S (the blood from which blood cells have been filtered out by the membrane filter 55) leaks out into the channel 52 by capillary action. Alternatively, in order to expedite reactions and to shorten detection time, a pump may be connected to the air aperture 54b, and the plasma S may be caused to flow by suctioning and extruding operations of the pump. In FIG. 14, the plasma S is represented by the hatched regions.

Step 4: The plasma S, which has leaked into the channel 52, and the labeling secondary antibodies C_{F}, are mixed together.

Step 5: the plasma S gradually flows along the channel 52 toward the air aperture 54b, and the antigens A and the labeling secondary antibodies C_{F} bind with the primary antibodies C₁, which are immobilized onto the first sensor portion 58′, in a competitive manner.

Step 6: A portion of the labeling secondary antibodies C_{F} that did not bind with the primary antibodies C₁ at the first sensor portion 58′ bind with the primary antibodies C₀, which are immobilized onto the second sensor portion 59′. Further, even in the case that the labeling secondary antibodies C_{F} which did not bind with the primary antibodies C₁ or the primary antibodies C₀ remain on the sensor portions, the following plasma S functions as a cleansing agent that washes the labeling secondary antibodies C_{F}, which are floating or non specifically bound onto the plate, away.

In this manner, the blood So is injected through the injection opening 54a, and step 1 through step 6 are performed to cause the antigens A and the secondary antibodies C₃ to bind with the primary antibodies C₁ on the first sensor portion 58′ in a competitive manner. Thereafter, detection signals are detected at the first sensor portion 58′ and the second sensor portion 59′, to detect the presence and/or the concentration of the antigens A. Thereafter, the detection sample cell 50B is moved in the X direction to enable signal detection from the second sensor portion 59′. The detection signals obtained at the second sensor portion 59′ are employed as a reference to calibrate the detection signals obtained at the first sensor portion 58′, to obtain more accurate detection results.

As described above, in the competition method, if the concentration of the antigens A is high, the amount of the labeling secondary antibodies C_{F} that bind with the primary antibodies C₁ is small. That is, the number of particles of the fluorescent substance F that bind with the primary antibodies C₁ is small, and therefore the fluorescent intensity is low. On the other hand, if the concentration of the antigens A is low, the amount of the labeling secondary antibodies C_{F} that bind with the primary antibodies C₁. That is, the number of particles of the fluorescent substance F that bind with the primary antibodies C₁ is great, and therefore the fluorescent intensity is high. The competition method enables measurement as long as the detection target substance has a single epitope. Therefore, the competition method is suited for detection of low molecular weight substances.

The detection sample cell 50B of the third embodiment is also equipped with the sensor chip having the sensor portions with the first electric charges on the surfaces thereof, and the fluorescent substance F having the fluorescent pigment molecules f enveloped therein and the second electric charges on the surfaces thereof. Accordingly, the same advantageous effects which are obtained by the detection sample cell of the first embodiment can also be obtained by the detection sample cell of the second embodiment.

### (Design Modifications to the Detection Sample Cell)

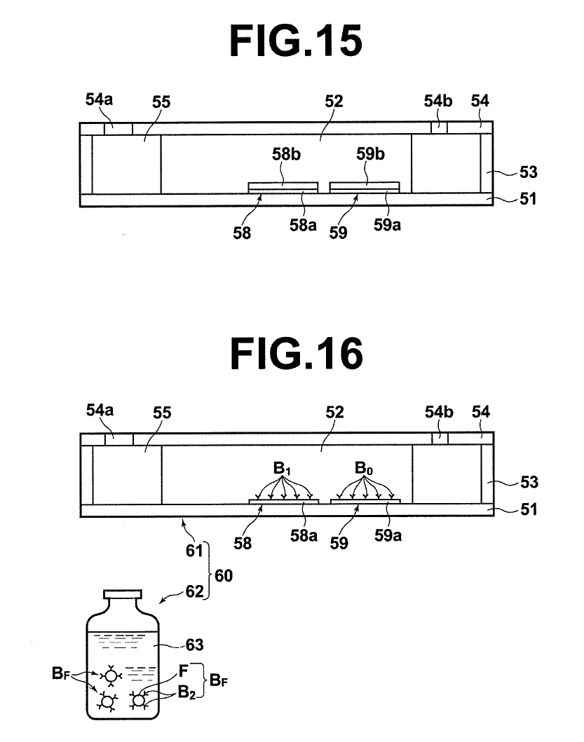

FIG. 15 is a sectional view that illustrates a detection sample cell to be employed in a detecting method and a detecting apparatus that utilizes optical field enhancement due to an optical waveguide mode. The detection sample cell of FIG. 15 differs from the detection sample cell according to the first embodiment illustrated in FIG. 10 in that optical waveguide layers 58b and 59b are provided on the metal layers 58a and 59a of the sensor portions, respectively. In this case, the functional groups that impart electric charges are immobilized on the optical waveguide layers 58b and 59b. For example, in the case that the optical waveguide layers are formed by SiO₂, electric charges can be imparted onto the surfaces of the sensor portions 58 and 59 by a silane coupling agent having functional groups at the ends thereof.

The detection sample cell may be utilized as the sensor chip of the detecting apparatus and the detecting method of the fourth and fifth embodiments, by immobilizing the aforementioned antibodies onto the optical waveguide layers 58b and 59b.

### [Detecting Kit]

A detecting kit which is utilized in the detecting method of the present invention will be described. FIG. 16 is a schematic diagram that illustrates the construction of a detecting kit 60.

The detecting kit 60 of the present invention is equipped with: a sample cell 61, the surfaces of two sensor portions having first electric charges thereon; and a labeling solution 63, which is caused to flow through a channel of the sample cell 61 simultaneously with a liquid sample, or after the liquid sample is caused to flow through the channel. Here, the labeling solution 63 includes labeling secondary antibodies B_{F} (fluorescent label binding substance) constituted by the fluorescent substance F, which is modified with the secondary antibodies B₂ (second binding substance) that specifically bind with the antigens A and has second electric charges opposite the first electric charges of the sensor portions.

The sample cell 61 differs from the sample cell 50A of the second embodiment described above only in the point that it does not include the labeling secondary antibody adsorption area, at which the fluorescent label binding substance B_{F} having functional groups that impart the second electric charges are physically adsorbed.

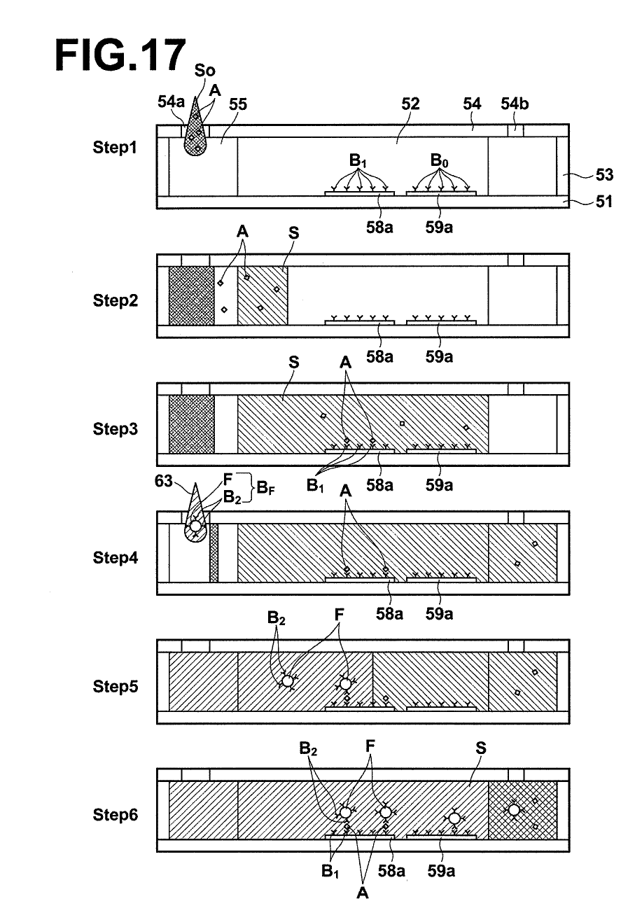

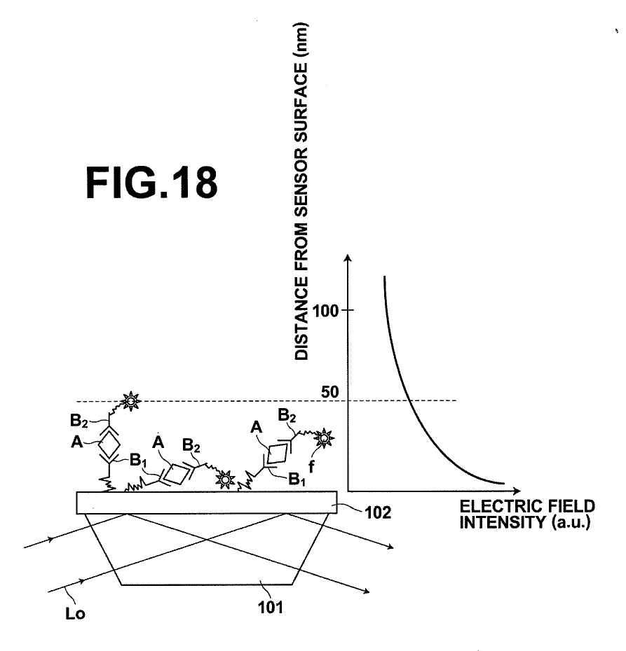

The procedures by which an assay is performed according to the sandwich method to detect whether an antigen to be detected is included in blood (whole blood) using the detecting kit 60 will be described with reference to FIG. 17.

Step 1: The blood So (whole blood), which is the target of inspection, is injected through the injection opening 54a. Here, a case will be described in which the blood So includes the antigen A to be detected. In FIG. 17, the blood So is represented by the cross hatched regions.

Step 2: The blood So is filtered by the membrane filter 55, and large molecules, such as red blood cells and white blood cells, are separated as residue. Plasma S (the blood from which blood cells have been filtered out by the membrane filter 55) leaks out into the channel 52 by capillary action. Alternatively, in order to expedite reactions and to shorten detection time, a pump may be connected to the air aperture 54b, and the plasma S may be caused to flow by suctioning and extruding operations of the pump. In FIG. 17, the plasma S is represented by the hatched regions.

Step 3: The plasma S gradually flows along the channel 52 toward the air aperture 54b, and the antigens A bind with the primary antibodies B₁, which are immobilized onto the first sensor portion 58.

Step 4: The labeling solution 63 that includes the labeling secondary antibodies B_{F}, is injected through the injection opening 54a.

Step 5: The labeling secondary antibodies B_{F} leaks out into the channel 52 by capillary action. Alternatively, in order to expedite reactions and to shorten detection time, a pump may be connected to the air aperture 54b, and the labeling solution 63 may be caused to flow by suctioning and extruding operations of the pump.

Step 6: The labeling secondary antibodies B_{F} gradually flow downstream, bind with the antigens A, and so called sandwich configurations, in which the antigens A are sandwiched between the primary antibodies B₁ and the labeling secondary antibodies B₂, are formed. A portion of the labeling secondary antibodies B_{F} that does not bind with the antigens binds with the primary antibodies B₀, which are immobilized on the second sensor portion 59. Further, even in the case that the labeling secondary antibodies B_{F} which did not bind with the primary antibodies B₁ or the primary antibodies B₀ remain on the measuring areas, the following plasma S functions as a cleansing agent that washes the labeling secondary antibodies B_{F}, which are floating or non specifically bound onto the plate, away.

In this manner, the blood So is injected through the injection opening 54a, and step 1 through step 6 are performed to bind the antigens A with the primary antibodies and the labeling secondary antibodies. Thereafter, fluorescent signals are detected at the first sensor portion 58 to detect the presence and/or the concentration of the antigens, in a state that the fluorescent substance F is drawn to the first sensor portion 58 due to static electric interactions. Next, the sample cell 61 is moved in the X direction so as to enable signal detection at the second sensor portion 59, and detection signals are detected at the second sensor portion 59. The fluorescent signals obtained at the second sensor portion 59, at which the primary antibodies B₀ that bind with the labeling secondary antibodies B_{F} are immobilized, are considered to be signals that reflect reaction conditions such as the amount of the labeling secondary antibodies B_{F} which has flowed through the channel 52 and the activity thereof. Therefore, if the detection signals obtained at the second sensor portion 59 are used as a reference to correct the fluorescent signals obtained at the first sensor portion 58, more accurate detection results can be obtained. Alternatively, a known amount of the labeling substance (fluorescent substance or fine metallic particles) may be immobilized onto the second sensor portion 59 in, and the detection signals obtained at the second sensor portion 59 may be used as a reference to correct the fluorescent signals obtained at the first sensor portion 58.

An example of a method for modifying the fluorescent substance F with the secondary antibodies, and a method for producing a labeling solution will be described.

A 50 mM MES buffer and a 5.0 mg/mL anti hCG monoclonal antibody solution (Anti hCG 5008 SP-5, by Medix Biochemica) are added to a fluorescent substance solution produced by the steps described previously (with a fluorescent substance having diameters of 500 nm, an excitation wavelength of 780 nm) and agitated. Thereby, fluorescent substances FB are modified with the antibodies.

Next, a 400 mg/mL ESC solution (01-62-0011 by Wako Pure Chemical Industries) is added, and the mixture is agitated at room temperature.

Further, a 2 mol/L glycine solution is added and the mixture is agitated. Thereafter, the mixture is subjected to centrifugal separation, to cause the particles to settle.

Finally, supernatant liquid is removed, PBS (having a pH of 7.4) is added, and the fluorescent substances are redispersed by an ultrasonic cleansing machine. Centrifugal separation is performed again, supernatant liquid is removed, 500 μL of a 1% BSA PBS solution (having a pH of 7.4) is added, and the fluorescent substances are redispersed, to obtain the labeling solution.

The detecting kit of the present invention is equipped with the sensor chip having the first electric charges on the surfaces of the sensor portions thereof, and the fluorescent substance F having fluorescent pigment molecules f enveloped therein and the second charges on the surfaces thereof. Accordingly, the amount of light which is generated due to excitation of the fluorescent labels can be easily detected in a state in which the fluorescent labels are drawn to the vicinities of the surfaces of the sensor portions where the electric field enhancing effect is great, due to static electric interactions. As a result, the electric field at the surface of the sensor portion, at which the degree of enhancement is great, can be efficiently utilized, and the distances from the surface of the sensor portion to the fluorescent labels can be uniformized. Accordingly, fluctuations in signal intensities can be suppressed. That is, stable signals having favorable S/N ratios can be detected, and the presence and/or the amounts of detection target substances can be accurately detected.

### (Design Modifications to the Detecting Kit)

By employing the detection sample cell illustrated in FIG. 13, the detecting kit of the present invention may be favorably used in a detecting apparatus and a detecting method that performs assays according to the competition method.

By employing the detection sample cell illustrated in FIG. 15 having the optical waveguide layer, the detecting kit of the present invention may be favorably used in a detecting apparatus and a detecting method that utilizes electric field enhancement dye to an optical waveguide mode.

What is claimed is:

1. A detecting method, comprising the steps of:
employing a sensor chip having a sensor portion provided on a surface of a dielectric plate;
causing a sample to contact the sensor portion, to cause an amount of a fluorescent label binding substance corresponding to the amount of a detection target substance included in the sample to bind onto the sensor portion;
irradiating an excitation light beam onto the sensor portion, to cause an enhanced optical field to be generated thereon;
exciting fluorescent labels of the fluorescent label binding substance with the enhanced optical field; and
detecting the amount of the detection target substance, based on the amount of light which is generated due to excitation of the fluorescent labels;
the sensor chip being that in which the sensor portion is of a laminated structure including a metal layer adjacent to the dielectric plate, and a first electric charge being present on the surface of the sensor portion; and
a fluorescent substance, having fluorescent pigment molecules which are enveloped in a light transmitting material that transmits fluorescence generated by the fluorescent pigment molecules, the surfaces of which are charged with second electric charges opposite the first electric charge being employed as the fluorescent labels.

2. A detecting method as defined in claim 1, wherein:
the first electric charge is imparted by a functional group, which is one of an acidic functional group and a basic functional group, corresponding to the charge; and
the second electric charges are imparted by the other of the acidic functional group and the basic functional group.

3. A detecting method as defined in claim 1, wherein:
the intensities of the first and second electric charges are controlled by controlling the pH of a solution on the sensor portion.

4. A detecting method as defined in claim 2, wherein:
the intensities of the first and second electric charges are controlled by controlling the pH of a solution on the sensor portion.

5. A detecting method as defined in claim 2, wherein:
the outermost surface layer of the laminated structure of the sensor chip which is employed is constituted by a self assembling film; and
functional groups which are included in the self assembling film are employed as the functional group that imparts the first electric charge onto the surface of the sensor portion

6. A detecting method as defined in claim 2, wherein:
the outermost surface layer of the laminated structure of the sensor chip which is employed is constituted by a polymer compound film; and
functional groups which are included in the polymer compound film are employed as the functional group that imparts the first electric charge onto the surface of the sensor portion.

7. A detecting method as defined in claim 6, wherein:
a blocking agent is employed as the polymer compound film.

8. A detecting method as defined in claim 1, wherein:
plasmon are excited at the metal layer by irradiating the excitation light beam, and the enhanced optical field is generated by the plasmon; and
fluorescence generated by the fluorescent labels due to excitation is detected as the light which is generated due to the excitation of the fluorescent labels.

9. A detecting method as defined in claim 1, wherein:
plasmon are excited at the metal layer by irradiating the excitation light beam, and the enhanced optical field is generated by the plasmon; and
radiant light that radiates toward the surface of the dielectric plate opposite the surface on which the sensor portion is provided from plasmon newly induced in the metal layer by fluorescence generated by the fluorescent labels due to excitation, is detected as the light which is generated due to the excitation of the fluorescent labels.

10. A detecting method as defined in claim 1, wherein:
the laminated structure of the sensor chip which is employed is equipped with an optical waveguide layer;
an optical waveguide mode is excited within the optical waveguide layer by irradiating the excitation light beam, and the enhanced optical field is generated by the optical waveguide mode; and
fluorescence generated by the fluorescent labels due to excitation is detected as the light which is generated due to the excitation of the fluorescent labels.

11. A detecting method as defined in claim 1, wherein:
the laminated structure of the sensor chip which is employed is equipped with an optical waveguide layer;
an optical waveguide mode is excited within the optical waveguide layer by irradiating the excitation light beam, and the enhanced optical field is generated by the optical waveguide mode; and
radiant light that radiates toward the surface of the dielectric plate opposite the surface on which the sensor portion is provided from plasmon induced at the metal layer by fluorescence generated by the fluorescent labels due to excitation, is detected as the light which is generated due to the excitation of the fluorescent labels.

12. A detecting apparatus to be employed to execute the detecting method defined in claim 1, comprising:
a housing portion for housing the sensor chip;
an excitation light beam irradiating optical system, for irradiating the excitation light beam onto the sensor portion;
light detecting means, for detecting the light which is generated due to excitation of the fluorescent labels by the enhanced optical field; and
pH control means, for controlling the pH of solutions on the sensor portion of the sensor chip which is housed in the housing portion.

13. A detection sample cell, to be utilized in the detecting method defined in claim 1, comprising:
a base having a channel through which liquid samples are caused to flow;
an injection opening provided at an upstream side of the channel for injecting the liquid samples into the channel;
an air aperture provided at a downstream side of the channel for causing the liquid samples which have been injected from the injection opening to flow downstream; and
a sensor chip portion provided within the channel between the injection opening and the air aperture, comprising a dielectric plate which is provided as a portion of an inner wall of the channel, and a sensor portion provided on a predetermined region of the dielectric plate on the sample contacting surface thereof;
the sensor portion being of a laminated structure that includes a metal layer adjacent to the dielectric plate; and
one of an acidic functional group and a basic functional group being present on the surface of the sensor portion.

14. A detection sample cell as defined in claim 13, further comprising:
a first binding substance, for immobilizing the fluorescent label binding substance onto the sensor portion, immobilized onto the sensor portion.

15. A detection sample cell as defined in claim 14, further comprising:
one of a second binding substance that specifically binds with the detection target substance and a third binding substance that specifically binds with the first binding substance and competes with the detection target substance; and
a fluorescent label binding substance modified with one of the second binding substance and third binding substance, having the one of an acidic functional group and a basic functional group, which is different from the functional group provided on the sensor portion, provided in the channel upstream from the sensor portion.

16. A detection sample cell as defined in claim 13, wherein the laminated structure is equipped with an optical waveguide layer.

17. A detecting kit to be utilized to execute the detecting method defined in claim 1, comprising:
a detection sample cell equipped with: a base having a channel through which liquid samples are caused to flow; an injection opening provided at an upstream side of the channel for injecting the liquid samples into the channel; an air aperture provided at a downstream side of the channel for causing the liquid samples which have been injected from the injection opening to flow downstream; a sensor chip portion provided within the channel between the injection opening and the air aperture, comprising a dielectric plate which is provided as a portion of an inner wall of the channel, and a sensor portion provided on a predetermined region of the dielectric plate on the sample contacting surface thereof; a first binding substance for immobilizing the fluorescent label binding substance onto the sensor portion, immobilized onto the sensor portion; the sensor portion being of a laminated structure that includes a metal layer adjacent to the dielectric plate; and one of an acidic functional group and a basic functional group being present on the surface of the sensor portion; and
a labeling solution which is caused to flow into the channel at one of a timing simultaneously with the liquid sample and a timing after the liquid sample is caused to flow into the channel, including a fluorescent substance modified with one of: a second binding substance that specifically binds with the detection target substance and a third binding substance that specifically binds with the first binding substance and competes with the detection target substance.

18. A detecting kit as defined in claim 17, wherein the laminated structure is equipped with an optical waveguide layer.
